(19) United States
(12) Patent Application Publication
(10) Pub. No.: US 2024/0269228 A1
(43) Pub. Date: Aug. 15, 2024

(54) SELECTIVE PENICILLAMINE SUBSTITUTION ENABLES DEVELOPMENT OF A POTENT ANALGESIC PEPTIDE THAT ACTS THROUGH A NON-OPIOID BASED MECHANISM

(71) Applicant: University of Utah Research Foundation, Salt Lake City, UT (US)

(72) Inventors: Joanna Gajewiak, Salt Lake City, UT (US); Joseph Michael McIntosh, Salt Lake City, UT (US)

(21) Appl. No.: 18/567,010

(22) PCT Filed: Mar. 16, 2022
(86) PCT No.: PCT/US2022/020613

§ 371 Date: Dec. 4, 2023

Related U.S. Application Data
(60) Provisional application No. 63/196,655, filed on Jun. 3, 2021.
(60) Provisional application No. 63/197,931, filed on Jun. 7, 2021.

(51) Int. Cl. A61K 38/10; A61P 29/00; C07K 7/08

(52) U.S. Cl. CPC A61K 38/10; A61P 29/00; C07K 7/08

(57) ABSTRACT

Disclosed herein are compositions and methods related to RgIA4 peptide analogs. An RgIA4 peptide analog can comprise a recognition finger region configured to bind to an α9α10 nicotinic acetylcholine receptor. The RgIA4 peptide analog can comprise at least two disulfide bridges comprising: an inter-cysteine disulfide bridge between a first cysteine and a second cysteine, and a penicillamine-cysteine disulfide bridge between a first penicillamine and a third cysteine. The RgIA4 peptide analog can have a binding affinity for the α9α10 nicotinic acetylcholine receptor that is at least 50% of a binding affinity of an RgIA4 peptide. A method of maintaining an RgIA4 peptide potency for an α9α10 nicotinic acetylcholine receptor in an RgIA4 peptide analog can comprise providing a composition as disclosed herein. A method for treating a condition responsive to α9α10 nicotinic acetylcholine receptor binding can comprise administering a therapeutically effective amount of the composition to the subject.

### RELATED APPLICATIONS

This application is a 371 U.S. national stage entry of PCT Application Ser. No. PCT/US2022/020613, filed Mar. 16, 2022, which claims the benefit of U.S. Provisional Patent Application Ser. No. 63/196,655, filed Jun. 3, 2021, and U.S. Provisional Patent Application Ser. No. 63/197,931, filed Jun. 7, 2021, the entirety of which are incorporated herein by reference.

### GOVERNMENT INTEREST

This invention was made with government support under grant numbers PO1 GM048677 and R01 GM103801 awarded by the National Institutes of Health. The government has certain rights in this invention.

### FIELD OF THE DISCLOSURE

The present disclosure relates to peptides and their analogs and therapeutic uses thereof. Accordingly, the present disclosure relates generally to the fields of biology, cell physiology, chemistry, pharmaceutical sciences, medicine, and other health sciences.

### BACKGROUND

Compounds produced by venomous predators are notable for their diverse structure, activity, and pharmacology. Minimal quantities of individual components may be sufficient to incapacitate prey or defend against a predator. A substantial fraction of venom components are small proteins and peptides: the latter are amenable to solid-phase synthesis and potential drug development. Examples include angiotensin converting enzyme inhibitors for hypertension (e.g., captopril), antiplatelet drugs for heart attack (e.g., eptifibatide), and glucagon-like peptide 1 (GLP-1) agonists (e.g. exenatide) for treatment of type-II diabetes.

Conus are predatory marine snails that hunt polychaete worms, mollusks and fish. There are over 830 known species of cone snails, each of which produces a unique cocktail of hundreds of distinct peptides targeted to a variety of receptors and ion channels. α-Conotoxins (α-Ctx), a subset of Conus peptides, are inhibitors of nicotinic acetylcholine receptors (nAChRs). nAChRs are pentameric ligand-gated ion channels with diverse receptor subtypes found in tissues, including neurons, immune cells, and skeletal muscle.

Over 53 million people worldwide use opioids. With over 450 deaths per day from opioid overdoses, finding non-opioid drugs to treat severe to chronic pain is desirable. Current clinically available medications treat pain but do not modify the underlying disease state.

### SUMMARY

In one embodiment, a synthetic analgesic peptide can comprise the amino acid sequence G C X1 T D P R C X2 (R-3-Y) Q C X3 X4 (SEQ ID NO: 3). In one aspect, X1 can be selected from the group consisting of L-penicillamine (L-Pen), D-penicillamine (D-Pen), and L-cysteine. In another aspect, X2 can be selected from the group consisting of L-arginine, D-arginine, or citrulline. In another aspect, X3 can be any amino acid. In another aspect, X4 can be any amino acid. In yet another aspect, R-3-Y can be 3-R-tyrosine. In one aspect, 3-R-tyrosine can be a peptide residue selected from the group consisting of 3-chloro-tyrosine, 3-fluoro-tyrosine, 3-iodo-tyrosine, 3-bromo-tyrosine, and tyrosine.

In another embodiment, an RgIA4 peptide analog can comprise: a recognition finger region configured to bind to an α9α10 nicotinic acetylcholine receptor. In one aspect, the RgIA4 peptide analog can comprise: at least two disulfide bridges comprising: an inter-cysteine disulfide bridge between a first cysteine and a second cysteine, and a penicillamine-cysteine disulfide bridge between a first penicillamine and a third cysteine. In another aspect, the RgIA4 peptide analog can have a binding affinity for the α9α10 nicotinic acetylcholine receptor that is at least 50% of a binding affinity of an RgIA4 peptide.

In yet another embodiment, a method of maintaining an RgIA4 peptide potency for an α9α10 nicotinic acetylcholine receptor in an RgIA4 peptide analog can comprise: providing an inter-cysteine disulfide bridge between a first cysteine and a second cysteine, and a penicillamine-cysteine disulfide bridge between a first penicillamine and a third cysteine. In another aspect, the method can comprise providing a β³-homo tyrosine bound to the third cysteine. In another aspect, the method can comprise providing a C-terminal residue selected from the group consisting of L-citrulline, D-citrulline, L-arginine, D-arginine, L-lysine, D-lysine, L-ornithine, and D-ornithine.

In one more embodiment, a method for treating in a subject, a condition that is responsive to α9α10 nicotinic acetylcholine receptor binding can comprise administering a therapeutically effective amount of a composition as disclosed herein to the subject.

There has thus been outlined, rather broadly, the more important features of the disclosure so that the detailed description thereof that follows may be better understood, and so that the present contribution to the art may be better appreciated. Other features of the present disclosure will become clearer from the following detailed description of the disclosure, taken with the accompanying drawings and claims, or may be learned by the practice of the disclosure.

### BRIEF DESCRIPTION OF THE DRAWINGS

Features and advantages of the disclosure will be apparent from the detailed description which follows, taken in conjunction with the accompanying drawings, which together illustrate, by way of example, features of the disclosure.

FIG. 1 illustrates stability of RgIA4 and RgIA-5474 in 25% human serum in accordance with an example. Top panel: HPLC traces of a mixture (˜1:1) of the globular and ribbon forms of the reference peptides RgIA4 and RgIA-5474. Bottom panel: serum stability at 24 h (n=6). The reaction was monitored by reverse-phase high-performance liquid chromatography (RP-HPLC), using a C18 column and the gradient: ranging from 10% to 50% of buffer B in 40 min, with a flow rate of 1mL/min.

FIG. 2 illustrates the effect of RgIA-5474 in oxaliplatin-induced peripheral neuropathy in mice in accordance with an example. Mice were injected i.p. with oxaliplatin (ox: 3.5 mg/kg) daily as described herein. Control animals were treated with vehicle. RgIA-5474 was dissolved in sterile saline (sal) and injected s.c. daily. Withdrawal latency was used as a measure of cold allodynia. The cold allodynia test was performed on days 8, 15, and 22 at 24 h after RgIA-5474 administration at 4 μg/kg (panel A) and 40 μg/kg (panel B). Values are expressed as the mean±SEM from eight mice for each experimental determination. ***P<0.001, **P<0.01, *P<0.05 significantly different from vehicle. FIG. 3 illustrates the evolution of RgIA with respect to human α9α10 nAChR in accordance with an example.

FIG. 4A illustrates an HPLC trace of the linear RgIA-5474 in accordance with an example. The HPLC trace was collected by RP-HPLC using C18 column and a linear gradient ranging from 10% to 50% buffer B in 40 min.

FIG. 4B illustrates an HPLC trace of the monocyclic RgIA-5474 in accordance with an example. The HPLC trace was collected by RP-HPLC using C18 column and a linear gradient ranging from 10% to 50% buffer B in 40 min.

FIG. 4C illustrates an HPLC trace of the fully-folded RgIA-5474 in accordance with an example. The HPLC trace was collected by RP-HPLC using a C18 column and a linear gradient ranging from 10% to 50% buffer B in 40 min.

FIG. 5A illustrates HPLC traces of fully folded peptide analogs in accordance with an example. Traces were collected at room temperature using analytical C18 Vydac RP-HPLC (218TP54, 250×4.6 mm, 5-μm particle size) and a gradient ranging from 10% B to 50% B in 40 min with a flow rate 1 mL/min using the following buffers: 0.1% (vol/vol) TFA in water (buffer A) and 0.092% TFA (vol/vol) in 60% aqueous acetonitrile (vol/vol) (buffer B). HPLC trace for RgIA-5493 was collected at 45° C. with the gradient ranging 10% B to 50% B in 40 min with a flow rate 1 mL/min with buffer B being 90% acetonitrile in aqueous 0.1% TFA.

FIG. 5B illustrates HPLC traces of fully folded peptide analogs in accordance with an example. Traces were collected using analytical C18 Vydac RP-HPLC (218TP54, 250×4.6 mm, 5-μm particle size) and a gradient ranging from 10% B to 50% B in 40 min with a flow rate 1 mL/min using the following buffers: 0.1% (vol/vol) TFA in water (buffer A) and 0.092% TFA (vol/vol) in 60% aqueous acetonitrile (vol/vol) (buffer B).

FIG. 6A illustrates concentration-response curves for the inhibition of human α9α10 nAChR in accordance with an example. Human α9α10 nAChRs were heterologously expressed in X. laevis oocytes and the potencies of the peptides assessed using two-electrode voltage clamp electrophysiology as described herein. Data points are the mean +SEM obtained from 3-5 oocytes.

FIG. 6B illustrates concentration-response curves for the inhibition of human α9α10 nAChR in accordance with an example. Human α9α10 nAChRs were heterologously expressed in X. laevis oocytes and the potencies of the peptides assessed using two-electrode voltage clamp electrophysiology as described herein. Data points are the mean +SEM obtained from 3-5 oocytes.

FIG. 6C illustrates concentration-response curves for the inhibition of human α9α10 nAChR in accordance with an example. Human α9α10 nAChRs were heterologously expressed in X. laevis oocytes and the potencies of the peptides assessed using two-electrode voltage clamp electrophysiology as described herein. Data points are the mean +SEM obtained from 3-5 oocytes.

FIG. 7 illustrates a concentration-response curve for the inhibition of human α9α10 nAChRs by RgIA-5711 in accordance with an example. Rat α9α10 nAChRs were heterologously expressed in X. laevis oocytes and the potency of the peptide assessed using two-electrode voltage clamp electrophysiology as described herein. Data points are the mean +SEM obtained from 3-5 oocytes.

FIG. 8 illustrates the stability of RgIA5 in 25% human serum in accordance with an example. Left panel: HPLC trace of the globular form of the reference RgIA5. Right panel: serum stability at 24 h (n=6). After 24 h there is 41% (+0.5) of the globular form present and 59% (+0.5) of the ribbon form. The reaction was monitored by RP-HPLC, using C18 column and the gradient ranging from 10% to 50% of buffer B in 40 min, with a flow rate of 1mL/min.

FIG. 9A illustrates a concentration-response curve for the inhibition of human α7 nAChR by RgIA-5474 in accordance with an example. Human α7 nAChRs were heterologously expressed in X. laevis oocytes and the potency of the peptide assessed using two-electrode voltage clamp electrophysiology as described herein. Data points are the mean±SEM obtained from 3-5 oocytes.

FIG. 9B illustrates a concentration-response curve for the inhibition of rat α9α10 nAChRs by RgIA-5474 in accordance with an example. Rat α9α10 nAChRs were heterologously expressed in X. laevis oocytes and the potency of the peptide assessed using two-electrode voltage clamp electrophysiology as described herein. Data points are the mean±SEM obtained from 3-5 oocytes.

These drawings are provided to illustrate various aspects of the disclosure and are not intended to be limiting of the scope in terms of dimensions, materials, configurations, arrangements, or proportions unless otherwise limited by the claims.

### DETAILED DESCRIPTION

While these exemplary embodiments are described in sufficient detail to enable those skilled in the art to practice the disclosure, it should be understood that other embodiments may be realized and that various changes to the disclosure may be made without departing from the spirit and scope of the present disclosure. Thus, the following more detailed description of the embodiments of the present disclosure is not intended to limit the scope of the disclosure, as claimed, but is presented for purposes of illustration only and not limitation to describe the features and characteristics of the present disclosure, to set forth the best mode of operation of the disclosure, and to sufficiently enable one skilled in the art to practice the disclosure. Accordingly, the scope of the present disclosure is to be defined solely by the appended claims.

### Definitions

In describing and claiming the present disclosure, the following terminology will be used.

The singular forms “a,” “an,” and “the” include plural referents unless the context clearly dictates otherwise. Thus, for example, reference to “a peptide” includes reference to one or more of such materials and reference to “facilitating” refers to one or more such steps.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility and imprecision associated with a given term, metric, or value. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this written description support for actual numerical values is provided even when the term “about” is used therewith. For example, the recitation of “about” 30 should be construed as not only providing support for values a little above and a little below 30, but also for the actual numerical value of 30 as well. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, the terms “treat.” “treatment,” or “treating” and the like refers to administration of a therapeutic agent or therapeutic action to a subject who is either asymptomatic or symptomatic. In other words, “treat,” “treatment,” or “treating” can refer to the act of reducing or eliminating a condition (i.e., symptoms manifested), or it can refer to prophylactic treatment (i.e., administering to a subject not manifesting symptoms in order to prevent their occurrence). Such prophylactic treatment can also be referred to as prevention of the condition, preventative action, preventative measures, and the like.

As used herein, the terms “therapeutic agent.” “active agent,” and the like can be used interchangeably and refer to agent that can have a beneficial or positive effect on a subject when administered to the subject in an appropriate or effective amount. In one aspect, the therapeutic or active agent can be an RgIA4 peptide analog. The terms “additional active agent,” “supplemental active agent,” “secondary active agent,” and the like can be used interchangeably and refer to a compound, molecule, or material other than be an RgIA4 peptide analog.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients. Furthermore, the term “dosage form” can include one or more formulation(s) or composition(s) provided in a format (e.g., a specific form, shape, vehicle, etc.) for administration to a subject. For example, an “oral dosage form” can be suitable for administration to a subject's mouth. A “topical dosage form” can be suitable for administration to a subject's skin by rubbing, or the like.

As used herein, a “treatment situs” refers to a location on or within a subject where treatment is desired. For example, when treating pain, the treatment situs can be the area of the pain. Further, as used herein, an “application situs” refers to a location on or in a subject where treatment is administered. Further, the application situs for an infusion dosage formulation may be an area where the infusion equipment enters the subject's circulatory system. Yet further, the application situs for a topical dosage formulation may be the area of skin or mucosa to which the topical dosage formulation is applied. In some embodiments, the application situs may be substantially the same as the treatment situs (e.g., the composition or formulation is administered directly to the treatment site). In other embodiments, the application situs may be different from (e.g., distal from) the treatment situs. In such cases, even though administration may be distal from the treatment situs, the composition or formulation still exerts a therapeutic effect at the treatment situs.

As used herein, “topical composition” or “topical administration” and the like refer to a composition suitable for administration directly to a skin or mucosa surface and from which an effective amount of a drug is released. In some embodiments, topical compositions can provide a local or localized therapeutic effect (e.g., at or near an application situs). For example, a topical composition when applied to a wound, a lesion, a burn, a canker sore, etc. (e.g., a treatment situs), may primarily exert a therapeutic effect at or around the application situs, but not substantially beyond it. In other embodiments, a topical composition can provide a regional effect. For example, a topical composition administered to a skin surface on a region of the body, such as a finger, arm, ankle, joint, etc. can exert a therapeutic effect within the region, but not substantially beyond. For example, a topical composition administered to the region of an ankle can have a therapeutic effect in and around the ankle, by for example, reducing edema, joint inflammation, pain, etc. In other embodiments, topical compositions can provide a systemic effect. In some aspects, a topical composition can provide the therapeutic effect though a mechanism of action where the drug or active agent itself arrives at the treatment situs. In other aspects, the topical composition can provide the therapeutic effect through an intermediate mechanism of action, such as biochemical cascade event, such as an enzymatic cascade or other signaling (e.g., cellular signaling, or inter/intra cellular signaling) event which ultimately exerts the desired therapeutic effect at the treatment situs. In some examples, such intermediate mechanism can allow treatment of a treatment situs that is distal from an application situs. In yet other examples, when treatment of a distal treatment situs occurs, the active agent may travel through dermal and other tissues from the application situs to the treatment situs and exert a direct effect.

As used herein, “transdermal” refers to the route of administration of a therapeutic agent through an unbroken skin surface when administered to the skin surface. When transdermally administered, the drug or active agent migrates from the application situs to a treatment situs and exerts a therapeutic effect. Transdermal compositions and dosage forms can include structures and/or devices which assist in holding the composition on a skin surface, such as, for example, backing films, adhesives, reservoirs, etc. Furthermore, transdermal compositions can include agents which aid or otherwise facilitate movement of the active agent from an application situs to a treatment situs (e.g., through the skin and into the subject's circulatory system) such as penetration or permeation enhancers. Such penetration or permeation enhancers can also be used with topical formulations in some embodiments.

The term “skin” or “skin surface” includes not only the outer skin of a subject comprising one or more epidermal layers, but also mucosal surfaces such as the mucosa of the respiratory (including nasal and pulmonary), oral (mouth and buccal), vaginal, and rectal cavities. Hence, the term “transdermal” may encompass “transmucosal” as well.

As used herein, “co-administering” a first therapeutic agent with a second therapeutic agent can include concomitant administration within a suitable time window. In one example, the suitable time window can be less than one or more of: 1 hour, 45 minutes, 30 minutes, 15 minutes, 5 minutes, 2 minutes, 1 minute, or a combination thereof. Concomitant administration can be from the same composition or from different compositions.

As used herein, a “subject” refers to a mammal that may benefit from the method or device disclosed herein. Examples of subjects include humans, and may also include other animals such as horses, pigs, cattle, dogs, cats, rabbits, and aquatic mammals. In one specific aspect, the subject is a human.

As used herein, a “dosing regimen” or “regimen” such as an “initial dosing regimen” or “starting dose” or a “maintenance dosing regimen” refers to how, when, how much, and for how long a dose of the compositions of the present disclosure can be administered to a subject. For example, an initial or starting dose regimen for a subject may provide for a total daily dose of from about 15 mcg/1 mL to about 1500 mcg/1 mL administered in two divided doses at least 12 hours apart (e.g., once with breakfast and once with dinner) with meals repeated daily for 30 days.

As used herein, “daily dose” refers to the amount of active agent (e.g., an RgIA4 peptide analog) administered to a subject over a 24-hour period of time. The daily dose can be administered two or more administrations during the 24-hour period. In one embodiment, the daily dose provides for two administrations in a 24-hour period. With this in mind, an “initial dose” or initial daily dose” refers to a dose administered during the initial regimen or period of a dosing regimen.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, an “acute” condition refers to a condition that can develop rapidly and have distinct symptoms needing urgent or semi-urgent care. By contrast, a “chronic” condition refers to a condition that is typically slower to develop and lingers or otherwise progresses over time. Some examples of acute conditions can include without limitation, an asthma attack, bronchitis, a heart attack, pneumonia, and the like. Some examples of chronic conditions can include without limitation, arthritis, diabetes, hypertension, high cholesterol, and the like.

As used herein, a recognition finger region is a region of a peptide that binds to a receptor. In one example, the recognition finger region of an RgIA4 peptide or RgIA4 analog can comprise Asp5-Pro6-Arg7.

As used herein, “selectivity” refers to modifying an action that provides a difference within a group (e.g., a group of cells) or between groups (e.g., a group of non-viable cells and a group of viable cells). For example, the action can be receptor binding and the groups can be a first receptor and a second receptor. For example, “selective receptor binding” of a first receptor compared to a second receptor can provide a difference between the first receptor and the second receptor at a selectivity ratio. In one example, the selectivity ratio differs from a 1:1 ratio. In one example, the selectivity ratio can be a ratio that is greater than at least one of: 1:1, 2:1, 3:1: 4:1, 5:1, 6:1, 7:1, 8:1, 9:1, 10:1, 15:1, 20:1, 25:1, 30:1, 35:1, 40:1, 45:1, 50:1, 100:1, the like, and a combination thereof.

As used herein, “stability” refers to the maintenance of an active agent in an active form. In one example, the stability of a peptide can be a measure of the concentration of the peptide in an active form after a selected time.

As used herein, “potency” refers to the concentration of an active agent that facilitates a selected response. In one example, the potency of a peptide (e.g., RgIA, RgIA4, or an RgIA4 analog) can be one or more of the half-maximal effective concentration (EC₅₀), half-maximal inhibitory concentration (IC₅₀), median effective dose (ED50), the like, or a combination thereof.

As used herein, a “binding affinity” of a peptide (e.g., RgIA, RgIA4, or an RgIA4 analog) for a receptor can be the concentration of peptide that would occupy 50% of the receptor in the absence of ligands. In one example, the binding affinity can be an inhibition constant (Kᵢ) that can be determined from the IC₅₀ using: Kᵢ=IC₅₀/([A]/EC₅₀)+1), wherein [A] is a fixed concentration of agonist, EC₅₀ is the concentration of agonist that results in half maximal activation of the receptor. In another example, ligand affinities can be measured directly as a dissociation constant (Kd) using methods such as fluorescence quenching, isothermal titration calorimetry, or surface plasmon resonance.

As used herein “D-substituted analogs” include RgIA and RgIA4 analogs disclosed herein having one or more L-amino acids substituted with D-amino acids. The D-amino acid can be the same amino acid type as that found in the analog sequence or can be a different amino acid. Accordingly, D-analogs are also Variants.

As used herein “Variants” include RgIA analogs disclosed herein wherein one or more amino acids have been replaced with a non-amino acid component, or where the amino acid has been conjugated to a functional group or a functional group has been otherwise associated with an amino acid. The modified amino acid may be, e.g., a glycosylated amino acid, a PEGylated amino acid (covalent and non-covalent attachment or amalgamation of polyethylene glycol (PEG) polymers), a farnesylated amino acid, an acetylated amino acid, an acylated amino acid, a biotinylated amino acid, a phosphorylated amino acid, an amino acid conjugated to a lipid moiety such as a fatty acid, or an amino acid conjugated to an organic derivatizing agent. The presence of modified amino acids can faciliate, for example, (a) increased polypeptide serum half-life and/or functional in vivo half-life, (b) reduced polypeptide antigenicity. (c) increased polypeptide storage stability. (d) increased peptide solubility. (e) prolonged circulating time, and/or (f) increased bioavailability, e.g., increasing the area under the curve (AUC). Amino acid(s) can be modified, for example, co-translationally or post-translationally during recombinant production (e.g., N-linked glycosylation at N-X-S/T motifs during expression in mammalian cells) or modified by synthetic means. The modified amino acid can be within the sequence or at the terminal end of a sequence. Variants can include derivatives as described elsewhere herein.

As used herein, “I-3-Y” or “iY” is 3-iodo-tyrosine, and “3-R-tyrosine” and “R-3-Y” is a peptide residue selected from the group consisting of 3-chloro-tyrosine, 3-fluoro-tyrosine, 3-iodo-tyrosine, 3-bromo-tyrosine, and tyrosine.

As used herein, “Cit” is citrulline.

As used herein, “b³hY” is beta-3-homo-tyrosine.

As used herein, “bhY” is beta-homo-tyrosine.

As used herein, “3-R-bhY” is a peptide residue selected from the group consisting of 3-chloro-beta-homo-tyrosine, 3-fluoro-beta-homo-tyrosine, 3-iodo-beta-homo-tyrosine, 3-bromo-beta-homo-tyrosine, and beta-homo-tyrosine.

As used herein, “3-R-b³hY” is a peptide residue selected from the group consisting of 3-chloro-beta-3-homo-tyrosine, 3-fluoro-beta-3-homo-tyrosine, 3-iodo-beta-3-homo-tyrosine, 3-bromo-beta-3-homo-tyrosine, and beta-3-homo-tyrosine.

As used herein, “Xaa” is any amino acid. Moreover, as used in this written description, Xaa provides express support for any amino acid. For example, Xaa provides express support for any amino acid or derivative thereof. For example, Xaa provides support for: alanine (Ala or A), arginine (Arg or R), asparagine (Asn or N), aspartic acid (Asp or D), cysteine (Cys or C), glutamic acid (Gly or E), glutamine (Gln or Q), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Tyr or W), Tyrosine (Tyr or Y), Valine (Val or V), selenosysteine (Sec or U), the like, or a combination thereof.

As used herein, “Variants of RgIA analogs” or “Variants of RgIA-4 analogs” disclosed herein include peptides having one or more amino acid additions, deletions, or substitutions, as compared to an RgIA peptide disclosed herein or an RgIA-4 peptide disclosed herein.

Embodiments disclosed herein include the RgIA analogs described herein as well as variants, D-substituted analogs, modifications, and derivatives of the RgIA analogs described herein. In some embodiments, variants, D-substituted analogs, modifications, and derivatives have 1, 2, 3, 4, 5, 6, 7, 8, 9, 10, 11, 12, 13, 14, or 15 sequence additions, deletions, substitutions, replacements, conjugations, associations, or permutations. Each analog peptide disclosed herein may also include additions, deletions, substitutions, replacements, conjugations, associations, or permutations at any position including positions 1, 2, 3, 4, 5, 6, 7, 8, 9, 10, 11, 12, 13, 14, or 15 of an analog peptide sequence disclosed herein.

In some embodiments an Xaa position can be included in any position of an analog peptide, wherein Xaa represents an addition, deletion, substitution, replacement, conjugation, association or permutation. In particular embodiments, each analog peptide has 1, 2, 3, 4, 5, 6, 7, 8, 9, 10, 11, 12, 13, 14, or 15 Xaa positions at one or more of positions 1. 2. 3, 4, 5, 6, 7, 8, 9, 10, 11, 12, 13, 14, or 15.

An analog can have more than one change (addition, deletion, substitution, replacement, conjugation, association, or permutation) and qualify as one or more of a variant, D-substituted analog, modification, and/or derivative. That is, inclusion of one classification of analog, variant, D-substituted analog, modification and/or derivative is not exclusive to inclusion in other classifications and all are collectively referred to as “analog peptides” herein.

An amino acid substitution can be a conservative or a non-conservative substitution. Variants of RgIA analogs disclosed herein can include those having one or more conservative amino acid substitutions. As used herein, a “conservative substitution” involves a substitution found in one of the following conservative substitutions groups: Group 1: alanine (Ala or A), glycine (Gly or G), serine (Ser or S), threonine (Thr or T): Group 2: aspartic acid (Asp or D), glutamic acid (Glu or E): Group 3: asparagine (Asn or N), glutamine (Gln or Q): Group 4: arginine (Arg or R), lysine (Lys or K), histidine (His or H): Group 5: isoleucine (Ile or I), leucine (Leu or L), methionine (Met or M), valine (Val or V): and Group 6: phenylalanine (Phe or F), tyrosine (Tyr or Y), tryptophan (Trp or W).

Additionally, amino acids can be grouped into conservative substitution groups by similar function, chemical structure, or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cys: acidic: Asp, Glu, Asn, and Gln: small aliphatic, nonpolar or slightly polar residues: Ala. Ser, Thr, Pro, and Gly: polar, negatively charged residues and their amides: Asp, Asn, Glu, and Gln: polar, positively charged residues: His, Arg, and Lys: large aliphatic, nonpolar residues: Met, Leu, Ile, Val, and Cys: and large aromatic residues: Phe, Tyr, and Trp. Additional information is found in Creighton (1984) Proteins, W.H. Freeman and Company.

As used herein, a “positive amino acid” includes the proteinogenic positive amino acids His, Arg, and Lys and the non-proteinogenic positive amino acids.

As used herein, an “aromatic amino acid” includes the proteinogenic aromatic amino acids Phe, Tyr, and Trp and the non-proteinogenic aromatic amino acids.

Variants of RgIA analogs or RgIA-4 analogs disclosed or referenced herein also include sequences with at least 70% sequence identity, at least 80% sequence identity, at least 85% sequence identity, at least 90% sequence identity, at least 95% sequence identity. at least 96% sequence identity, at least 97% sequence identity, at least 98% sequence identity, or at least 99% sequence identity to a peptide sequence disclosed or referenced herein. More particularly. variants of the RgIA analogs or RgIA-4 analogs disclosed herein include peptides that share: 70% sequence identity with any of SEQ ID NOs: 1-11: 80% sequence identity with any of SEQ ID NOs : 1-11: 81% sequence identity with any of SEQ ID NOs: 1-11: 82% sequence identity with any of SEQ ID NOs: 1-11: 83% sequence identity with any of SEQ ID NOs: 1-11: 84% sequence identity with any of SEQ ID NOs: 1-11: 85% sequence identity with any of SEQ ID NOs: 1-11: 86% sequence identity with any of SEQ ID NOs: 1-11: 87% sequence identity with any of SEQ ID NOs: 1-11: 88% sequence identity with any of SEQ ID NOs: 1-11: 89% sequence identity with any of SEQ ID NOs: 1-1: 90% sequence identity with any of SEQ ID NOs: 1-11: 91% sequence identity with any of SEQ ID NOs: 1-11: 92% sequence identity with any of SEQ ID NOs: 1-11: 93% sequence identity with any of SEQ ID NOs: 1-11: 94% sequence identity with any of SEQ ID NOs: 1-11: 95% sequence identity with any of SEQ ID NOs: 1-11: 96% sequence identity with any of SEQ ID NOs: 1-11: 97% sequence identity with any of SEQ ID NOs: 1-11: 98% sequence identity with any of SEQ ID NOs: 1-11: or 99% sequence identity with any of SEQ ID NOs: 1-11.

The C-terminus of a synthetic analgesic peptide may be a carboxylic acid or an amide group. The present disclosure also relates to RgIA analogs further modified by (i) additions made to the C-terminus, such as tyrosine. 3-iodo-tyrosine, a fluorescent tag, lipids. carbohydrates, or beta-homo amino acids. D/L-sulfono-γ-AApeptides. L-γ-AApeptides. and/or (ii) additions made to the N-terminus, such as tyrosine, 3-iodo-tyrosine. pyroglutamate, a fluorescent tag, lipids, carbohydrates, or beta-homo amino acids.

As used herein, the term “gene” refers to a nucleic acid sequence that encodes a peptide. This definition includes various sequence polymorphisms, mutations, and/or sequence variants wherein such alterations do not affect the function of the encoded peptide. The term “gene” may include not only coding sequences but also regulatory regions such as promoters, enhancers, and termination regions. “Gene” further can include all introns and other DNA sequences spliced from the mRNA transcript, along with variants resulting from alternative splice sites. Nucleic acid sequences encoding the peptide can be DNA or RNA that directs the expression of the peptide. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type. Gene sequences to encode peptides disclosed herein are available in publicly available databases and publications.

As used herein, recitation of a specific amino acid also includes support for the specific amino acid and any analog, variant, D-substituted analog, modification and/or derivatives thereof. In one example, recitation of tyrosine also explicitly includes support for 3-chloro-tyrosine, 3-fluoro-tyrosine, 3-iodo-tyrosine, tyrosine, ortho-tyrosine, 3-nitro-tyrosine, 3-amino-tyrosine, O-methyl-tyrosine, 2,6-dimethyl-tyrosine, beta-homo-tyrosine, Boc-Tyr(3,5-12)-OSu. [CpRu(Fmoc-tyrosin)]CF₃CO₂, O-(2-Nitrobenzyl)-L-tyrosine hydrochloride, 3-Nitro-L-tyrosine ethyl ester hydrochloride, N-(2,2,2-trifluoromethyl)-L-Tyrosine Ethyl Ester, DL-o-Tyrosine, the like, or a combination thereof. In one example, recitation of cysteine also explicitly includes support for cysteine, L-cysteic acid monohydrate, L-cysteinesulfinic acid monohydrate, seleno-L-cystine, the like, or a combination thereof. In one example, recitation of lysine also explicitly includes support for Fmoc-Lys(Me,Boc)-OH, Fmoc-Lys(Me)₃-OH Chloride, Fmoc-L-Lys(Nvoc)-OH, Fmoc-Lys(palmitoyl)-OH, Fmoc-L-Photo-Lysine, DL-5-Hydroxylysine hydrochloride, H-L-Photo-lysine HCl, the like, or a combination thereof.

In this disclosure, “comprises.” “comprising.” “containing” and “having” and the like can have the meaning ascribed to them in U.S. Patent law and can mean “includes.” “including.” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” “improved,” “maximized,” “minimized,” and the like refer to a property of a device, component, composition, biologic response, biologic status, or activity that is measurably different from other devices, components, compositions, biologic responses, biologic status, or activities that are in a surrounding or adjacent area, that are similarly situated, that are in a single device or composition or in multiple comparable devices or compositions, that are in a group or class, that are in multiple groups or classes, or as compared to an original (e.g. untreated) or baseline state, or the known state of the art. For example, an α-RgIA4 analog with “improved” performance in reducing neurologic pain would present an improvement with respect to at least one aspect of stability, binding efficacy, potency, or other performance related property as compared to other α-RgIA4 analogs.

The term “coupled,” as used herein, is defined as directly or indirectly connected in a chemical, mechanical, electrical or nonelectrical manner. As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only C, and combinations of each.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited: and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Occurrences of the phrase “in one embodiment,” or “in one aspect,” herein do not necessarily all refer to the same embodiment or aspect. Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### Example Embodiments

An initial overview of disclosure embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Nicotinic acetylcholine receptors (nAChRs), distributed throughout both peripheral and central nervous systems, are a group of transmembrane ligand-gated cationic channels that mediate fast synaptic transmission and are involved in a wide range of nervous system disorders including neuropathic pain, Parkinson's disease, schizophrenia, alcohol, and drug addiction. Different nAChR subunits including α, β, γ, δ, and ϵ associate in various combinations within these homo- or hetero-pentameric receptors, leading to a complex variety of nAChR subtypes with distinct pharmacological and biophysical functions. The nAChRs have previously been targeted for analgesic drug discovery albeit with progress being hindered by a narrow therapeutic window and side effects caused by indiscriminate subtype targeting.

Venom-derived compounds are of broad interest in neuropharmacology and drug development. α-Conotoxins are small disulfide-containing peptides from Conus snails that target nicotinic acetylcholine receptors (nAChRs) and are in clinical development for non-opioid based treatment of intractable pain. Although refined by evolution for interaction with target prey receptors, enhancements of pharmacological properties are used for use in mammalian systems.

In animal models, the cone snail venom derived peptides α-Ctx Vc1, RgIA, and GeXIVA can produce analgesic activity mediated via α9α10 nAChRs. The analgesia elicited by the second-generation analog, RgIA4, can last for several weeks after the treatment had been stopped. These findings indicate that the α9α10 nAChRs might be promising targets for the development of medications for pain and that Conus-derived peptides might generate potent analgesics.

Although promising in animal models, native α-conotoxins generally show low potency for human nAChRs. Recent studies have identified inhibition of the α9α10 nicotinic acetylcholine receptor (nAChR) subtype as a potential non-opioid based mechanism for chemotherapy-induced neuropathic pain.

RgIA is a 13 amino acid, positively charged, disulfide-rich peptide expressed in the venom of the marine snail Conus regius. The bioactive form of the peptide has a globular disulfide bond connectivity (2Cys-8Cys, 3Cys-12Cys), potently blocks rat α9α10 nAChRs (IC₅₀)=1.49 nM) but shows a 300-fold decrease in potency on human α9α10 nAChRs. The disulfide bridges may be used for the overall structure stabilizing effect and for peptide interaction with the channel. A large decrease in activity was observed when either one of the RgIA cysteine bridges was replaced with a dicarba bridge. Trans- and cis-[3,12]-dicarba RgIA analogs exhibited over 600-fold loss of potency in blocking rat α9α10 nAChRs with ICsos of 1.15 μM and 1.47 μM respectively. No activity on α9α10 nAChRs was observed for both cis and trans analogs when such replacement was made for the [2,8]-disulfide bridge. A similar effect was observed for α-Ctx Vc1.1, a potent antagonist of α9α10 nAChRs and γ-Aminobutyric acid type B receptors (GABABRs).

Among the α9α10 nAChR antagonists, the second generation analogue α-RgIA4, modified from the parent sequence α-RgIA, crosses over the “species-related affinity gap” and exhibits high potency for both rodent (IC_{50 0.9} nM) and human @9a10 nAChR (IC₅₀ 1.5 nM) without inhibiting other subtypes and other pain-related receptors (e.g., selectivity>1000 fold). Therefore, α-RgIA4 has potential as a lead compound for non-opioid analgesic development.

However, as with other disulfide-rich peptide drug molecules, α-RgIA4 is a poor candidate because of its low protease resistance and short plasma half-life. To address this, RgIA4 analogs can be synthesized that can include penicillamine and other non-proteinogenic amino acids. Some of the RgIA4 analogs had picomolar affinities that were an order of magnitude more potent than the parent RgIA4 analog. The highest affinity analog, RgIA-5474, had high selectivity, increased serum stability compared to RgIA4, and potent analgesic activity. That is, RgIA-5474 had a 30× increased potency on the human α9α10 nAChR and increased resistance to disulfide shuffling compared to the RgIA4 peptide. In addition, RgIA-5474 potently blocked the a9a10) nAChR, but not opioid- or other pain-related targets. In addition, RgIA-5474 effectively reversed chemotherapy-induced neuropathic pain.

In one embodiment, an RgIA4 peptide analog can include a recognition finger region configured to bind to an α9α10 nicotinic acetylcholine receptor, and at least two disulfide bridges including an inter-cysteine disulfide bridge between a first cysteine and a second cysteine and a penicillamine-cysteine disulfide bridge between a first penicillamine and a third cysteine. In one aspect, the RgIA4 peptide analog can have a binding affinity for the α9α10 nicotinic acetylcholine receptor that is at least 50% of a binding affinity of an RgIA4 peptide.

In another embodiment, a synthetic analgesic peptide can comprise the amino acid sequence G C XI T D P R C X2 (R-3-Y) Q C X3 X4 (SEQ ID NO: 3). In one aspect, X1 can be selected from the group consisting of L-penicillamine (L-Pen), D-penicillamine (D-Pen), and L-cysteine. In another aspect, X2 can be selected from the group consisting of L-arginine, D-arginine, or citrulline. In another aspect, X3 can be any amino acid. In another aspect, X4 can be any amino acid. In yet another aspect, R-3-Y can be 3-R-tyrosine.

In yet another embodiment, a method of maintaining an RgIA4 peptide potency for an α9α10 nicotinic acetylcholine receptor in an RgIA4 peptide analog can include providing an inter-cysteine disulfide bridge between a first cysteine and a second cysteine, and a penicillamine-cysteine disulfide bridge between a first penicillamine and a third cysteine. In another aspect, the method can include providing a β³-homo tyrosine bound to the third cysteine. In another aspect, the method can include providing a C-terminal residue selected from the group consisting of L-citrulline, D-citrulline, L-arginine, D-arginine, L-lysine, D-lysine, L-ornithine, and D-ornithine.

In another embodiment, a method for treating in a subject, a condition that is responsive to α9α10 nicotinic acetylcholine receptor binding, can comprise administering a therapeutically effective amount of a composition as disclosed herein to the subject.

### Analogs of α-RgIA4

Analogs of RgIA4 peptides can include various subregions that can be modified to enhance the stability, binding affinity, solubility, potency, selectivity, or the like when compared to an RgIA4 peptide without the modifications. For example, an RgIA4 peptide analog can include a recognition finger region that can be configured to bind to an α9α10 nicotinic acetylcholine receptor. In one example, the recognition finger region can be the region of the RgIA4 peptide analog that includes L-aspartic acid in the fifth position, L-proline in the sixth position, and L-arginine in the seventh position.

The stability and potency of the RgIA4 peptide can be reduced in human serum. Substitution with Pen can lead to widely varying effects depending on which of the four Cys residues is substituted. Three of the four positions can lead to losses in activity. In contrast, substitution of 3Cys can preserve the activity.

Thus, in one aspect, an RgIA4 peptide analog can include at least two disulfide bridges comprising: (a) an inter-cysteine disulfide bridge between a first cysteine (e.g., in the second position) and a second cysteine (e.g., in the eighth position), and (b) a penicillamine-cysteine disulfide bridge between a first penicillamine (e.g., in the third position) and a third cysteine (e.g., in the twelfth position). When the cysteine in the third position in a RgIA4 peptide is substituted by penicillamine, the stability and the potency of the RgIA4 peptide analog can be increased relative to the stability of the RgIA4 peptide.

In one aspect, the penicillamine-cysteine disulfide bridge can reduce one or more of disulfide bridge scrambling, disulfide bridge degradation, the like, or a combination thereof as compared to one or more of an RgIA4 peptide, an RgIA4 peptide analog without a penicillamine-cysteine disulfide bridge, the like, or a combination thereof.

In another aspect, the penicillamine-cysteine disulfide bridge can facilitate a stability for the RgIA4 peptide analog in human serum that is greater than the stability of an RgIA4 peptide in human serum. In one aspect, the stability in the human serum can be measured by the amount of a globular form of the RgIA4 peptide analog remaining after incubation of the RgIA4 peptide analog in 25% human serum AB type and incubated at 37° C. for at least one or more of 30 minutes, 1 hour, 2 hours, 4 hours, 8 hours, 24 hours, 48 hours, 72 hours, one week, two weeks, the like, or a combination thereof. In one aspect, the stability in human serum of the RgIA4 peptide analog can be at least one or more of 10%, 20%, 40%, 60%, 80%, 100%, 200%, 300%, 400%, 500%, or 1000% greater than the stability of the RgIA4 peptide in human serum.

The penicillamine-cysteine disulfide bridge can also facilitate a stability for the RgIA4 peptide analog in reduced glutathione that is greater than the stability of an RgIA4 peptide in reduced glutathione. In one aspect, the stability in the reduced glutathione can be measured by the amount remaining after incubation of 0.1 mg/ml of the RgIA4 peptide analog or the RgIA4 peptide in 10 equivalents of reduced glutathione in phosphate buffered saline (PBS) having a pH of 7.4 and incubated at 37° C.for at least one or more of 30 minutes, 1 hour, 2 hours, 4 hours, 8 hours, 24 hours, 48 hours, 72 hours, one week, two weeks, the like, or a combination thereof. In one aspect, the stability in the reduced glutathione of the α-RgIA4 peptide analog can be at least one or more of 10%, 20%, 40%, 60%, 80%, 100%, 200%, 300%, 400%, 500%, 1000%, the like, or a combination thereof greater than the stability of the RgIA4 peptide in the reduced glutathione.

The storage stability of the α-RgIA4 peptide analog can be enhanced when compared to the storage stability of the α-RgIA4 peptide. In one example, the storage stability can be measured when stored for a selected storage time at ambient humidity and temperature. In some cases, a storage time of greater than one or more of 1 day, 1 week, 2 weeks, 4 weeks, 3 months, 6 months, one year, or combinations thereof can be measured to compare enhancements in stability between the α-RgIA4 peptide analog and the α-RgIA4 peptide.

The maintenance of the RgIA4 peptide analog in a globular form can enhance the binding affinity for the α9α10 nicotinic acetylcholine receptor when compared to the binding affinity of the RgIA4 peptide for the α9α10 nicotinic acetylcholine receptor. In one example, the RgIA4 peptide analog can have a binding affinity for the α9α10 nicotinic acetylcholine receptor that is at least 50% of a binding affinity of an RgIA4 peptide. In one aspect, the binding affinity for the α9α10 nicotinic acetylcholine receptor can be substantially equal to the binding affinity of the RgIA4 peptide, greater than the binding affinity of an RgIA4 peptide, or the like.

The binding affinity of the RgIA4 peptide analog can be related to an IC₅₀ value of the RgIA4 peptide analog as provided by the Cheng-Prusoff equation. In one aspect, the RgIA4 peptide analog can provide an α9α10 nicotinic acetylcholine receptor IC₅₀ value that is at least 25.0× less than the α9α10 nicotinic acetylcholine receptor IC₅₀ value of the RgIA4 peptide, or at least 10.0× less than the α9α10 nicotinic acetylcholine receptor IC₅₀ value of the RgIA4 peptide, or at least 5.0× less than the @9a 10 nicotinic acetylcholine receptor IC₅₀ value of the RgIA4 peptide, or at least 2.0× less than the α9α10 nicotinic acetylcholine receptor IC₅₀ value of the RgIA4 peptide, or substantially equal to an α9α10 nicotinic acetylcholine receptor IC₅₀ value of the RgIA4 peptide, or no greater than 2.0× the α9α10 nicotinic acetylcholine receptor IC₅₀ value of the RgIA4 peptide, or no greater than 3.0× the α9α10 nicotinic acetylcholine receptor IC₅₀ value of the RgIA4 peptide, no greater than 5.0× the α9α10 nicotinic acetylcholine receptor IC₅₀ value of the RgIA4 peptide, the like, or a combination thereof.

The RgIA4 peptide analog can be modified in other ways to enhance the stability. binding affinity, solubility, potency, selectivity, or the like when compared to an RgIA4 peptide without the modifications. β-amino acids are homologs of natural α-amino acids, with an extra methylene group immediately before the carboxylic group in the backbone of the amino acids. The side chain can be positioned either on the α- or β-carbon (β2- or β3-analog, respectively), or can be present in both positions to maintain potency and provide increased resistance to proteolysis. In one aspect, the RgIA4 peptide analog can comprise a β³-homo tyrosine in the thirteenth position that can be bound to the cysteine in the twelfth position.

The RgIA4 peptide analog can have other modifications that enhance the stability. binding affinity, solubility, potency, selectivity, or the like when compared to an RgIA4 peptide without the modifications. The incorporation of Pen residues can lead to increased hydrophobicity and consequent precipitation. The addition of a positively charged residues at the C-terminal position can increase the RgIA4 peptide analog solubility without substantially affecting the RgIA4 peptide analog's potency. In one aspect, the RgIA4 peptide analog can comprise a C-terminal residue selected from the group consisting of L-citrulline, D-citrulline, L-arginine, D-arginine, L-lysine, D-lysine, L-ornithine, and D-ornithine. In one aspect, the RgIA4 peptide analog can have a solubility that is at least one or more of: 5×, 10×, 20×, 50×, 100×, or 200×, 500×, or 1000× greater than the solubility of the RgIA4 peptide analog without the C-terminal residue.

In one aspect, the RgIA4 peptide analog can facilitate an α9α10 nicotinic acetylcholine receptor selectivity that can be substantially equal to the α9α10 nicotinic acetylcholine receptor selectivity of an RgIA4 peptide. In one example, the RgIA4 peptide analog can facilitate an α9α10 nicotinic acetylcholine receptor selectivity that is at least one or more of 5×, 10×, 20×, 50×, 100×, or 200×, 500×, or 1,000× more selective for the α9α10 nicotinic acetylcholine receptor compared to a selectivity of a different nicotinic acetylcholine receptor (nAChR) subtype. In one aspect, the different nAChR subtype can be selected from the group consisting of: α1β1δϵ, α2β2, α2β4, α3β2, α3β4 α4β2, α4β4, α6/α3β2β3, α6/α3β4, α7, or a combination thereof.

When modified from the RgIA4 peptide, the safety profile of the RgIA4 peptide analog can be maintained or enhanced compared to a safety profile of the RgIA4 peptide. In one aspect, the RgIA4 peptide analog can have a safety profile that is substantially equal to or greater than the safety profile of an RgIA4 peptide. In one example, the safety profile can be measured by one or more aspects. In one aspect, the analog when present in a concentration of 100 μM can inhibit less than 25% of the human ether-a-go-go-related gene (hERG) K⁺ channel as measured from an automated-whole cell patch-clamp assay. In another aspect, the analog when present in a concentration of 10 μM can have inhibitory activity of less than 20% as measured in a CYP assay.

In one aspect, the RgIA4 peptide analog can comprise the amino acid sequence G C (Pen) T D P R C X5 ^{I3}Y Q C β³hY X6 (SEQ ID NO: 10). In one aspect, X5 can be selected from the group consisting of L-citrulline, D-citrulline, L-arginine D-arginine, the like, or a combination thereof. In another aspect, X6 can be selected from the group consisting of L-arginine, D-arginine, the like, or a combination thereof.

In another aspect, the RgIA4 peptide analog can comprise the amino acid sequence GC (Pen) T D P R C X5 ^{I3}Y Q C 3-R-Bβ³hY X6 (SEQ ID NO: 11). In one aspect, X5 can be selected from the group consisting of L-citrulline, D-citrulline, L-arginine D-arginine, the like, or a combination thereof. In another aspect, X6 can be selected from the group consisting of L-arginine, D-arginine, the like, or a combination thereof. In another aspect, 3-R-β³hY can be a peptide residue selected from the group consisting of 3-chloro-beta-3-homo-tyrosine, 3-fluoro-beta-3-homo-tyrosine, 3-iodo-beta-3-homo-tyrosine, 3-bromo-beta-3-homo-tyrosine, and beta-3-homo-tyrosine.

In another example, a synthetic analgesic peptide can comprise the amino acid sequence G C XI T D P R C X2 (R-3-Y) Q C X3 X4 (SEQ ID NO: 3). In one aspect, X1 can be selected from the group consisting of L-penicillamine (L-Pen), D-penicillamine (D-Pen), and L-cysteine. In another aspect, X2 can be selected from the group consisting of L-arginine, D-arginine, or citrulline. In another aspect, X3 can be any amino acid and X4 can be any amino acid. In another aspect, R-3-Y can be 3-R-tyrosine. In one example, R-3-tyrosine and R-3-Y can refer to a peptide residue selected from the group consisting of 3-chloro-tyrosine, 3-fluoro-tyrosine, 3-iodo-tyrosine, 3-bromo-tyrosine, and tyrosine.

In one example, X1 can be L-Pen, X2 can be L-arginine, X3 can be selected from the group consisting of beta-homo-tyrosine, 3-R-beta-homo-tyrosine (3-R-bhY), L-tyrosine, and D-tyrosine, and X4 can be selected from the group consisting of L-arginine and D-arginine. In one example, 3-R-bhY can be a peptide residue selected from the group consisting of 3-chloro-beta-homo-tyrosine, 3-fluoro-beta-homo-tyrosine, 3-iodo-beta-homo-tyrosine, 3-bromo-beta-homo-tyrosine, and beta-homo-tyrosine. In one example, beta-homo-tyrosine can be beta-3-homo-L-tyrosine. In another example, beta-homo-tyrosine can be beta-2-homo-L-tyrosine.

In another example, a synthetic analgesic peptide can comprise the amino acid sequence G C L-Pen T D P R C R (R-3-Y) QC X3 X4 (SEQ ID NO: 4). In this example, X3 can be any amino acid, X4 can be any amino acid, R-3-Y can be 3-R-tyrosine, wherein R-3-Y can refer to a peptide residue selected from the group consisting of 3-chloro-tyrosine, 3-fluoro-tyrosine, 3-iodo-tyrosine, 3-bromo-tyrosine, and tyrosine.

In another example, a synthetic analgesic peptide can comprise the amino acid sequence G C L-Pen T D P R C R (1-3-Y) QC X3 X4 (SEQ ID NO: 5). In this example,

X3 can be any amino acid, X4 can be any amino acid, and I-3-Y can be 3-iodo-L-tyrosine.

In another example, a synthetic analgesic peptide can comprise the amino acid sequence G C L-Pen T D P R C R (1-3-Y) Q C (bhY) X4 (SEQ ID NO: 6). In this example, X4 can be any amino acid, I-3-Y can be 3-iodo-L-tyrosine, and bhY can be beta-homo-tyrosine. In one example bhY can be beta-3-homo-L-tyrosine. In another example, bhY can be beta-2-homo-L-tyrosine.

In another example, a synthetic analgesic peptide can comprise the amino acid sequence G C L-Pen T D P R C R (1-3-Y) Q C (3-R-bhY) X4 (SEQ ID NO: 7). In this example, X4 can be any amino acid, I-3-Y can be 3-iodo-L-tyrosine, and 3-R-bhY can be a peptide residue selected from the group consisting of 3-chloro-beta-homo-tyrosine, 3-fluoro-beta-homo-tyrosine, 3-iodo-beta-homo-tyrosine, 3-bromo-beta-homo-tyrosine, and beta-homo-tyrosine. In one example bhY can be beta-3-homo-L-tyrosine. In another example, bhY can be beta-2-homo-L-tyrosine.

In another example, a synthetic analgesic peptide can comprise the amino acid sequence G C L-Pen T D P R C R (1-3-Y) Q C (bhY) R (SEQ ID NO: 8). In this example, 1-3-Y can be 3-iodo-L-tyrosine, and bhY can be beta-homo-tyrosine. In one example bhY can be beta-3-homo-L-tyrosine. In another example, bhY can be beta-2-homo-L-tyrosine.

In another example, a synthetic analgesic peptide can comprise the amino acid sequence G C L-Pen T D P R CR (1-3-Y) Q C (3-R-bhY) R (SEQ ID NO: 9). In this example, I-3-Y can be 3-iodo-L-tyrosine, and 3-R-bhY can be a peptide residue selected from the group consisting of 3-chloro-beta-homo-tyrosine, 3-fluoro-beta-homo-tyrosine, 3-iodo-beta-homo-tyrosine, 3-bromo-beta-homo-tyrosine, and beta-homo-tyrosine. In one example bhY can be beta-3-homo-L-tyrosine. In another example, bhY can be beta-2-homo-L-tyrosine.

In another embodiment, a method of maintaining an RgIA4 peptide potency for an α9α10 nicotinic acetylcholine receptor in an RgIA4 peptide analog can comprise providing an inter-cysteine disulfide bridge between a first cysteine and a second cysteine, and a penicillamine-cysteine disulfide bridge between a first penicillamine and a third cysteine. In one aspect, the method can comprise providing a B3-homo tyrosine bound to the third cysteine. In another aspect, the method can comprise providing a C-terminal residue selected from the group consisting of L-citrulline, D-citrulline, L-arginine, D-arginine, L-lysine, D-lysine, L-ornithine, and D-ornithine.

In one aspect, the method can comprise inhibiting the α9α10 nicotinic acetylcholine receptor with a selected IC₅₀ value. In one example, the RgIA4 peptide analog can inhibit the α9α10 nicotinic acetylcholine receptor with an IC₅₀ value that is: at least 25.0× less than the α9α10 nicotinic acetylcholine receptor IC₅₀ value of the RgIA4 peptide, or at least 10.0× less than the α9α10 l nicotinic acetylcholine receptor IC₅₀ value of the RgIA4 peptide, or at least 5.0× less than the α9α10 nicotinic acetylcholine receptor IC₅₀ value of the RgIA4 peptide, or at least 2.0× less than the α9α10 nicotinic acetylcholine receptor IC₅₀ value of the RgIA4peptide, or substantially equal to an α9α10 nicotinic acetylcholine receptor IC₅₀ value of the RgIA4 peptide, or no greater than 2.0× the α9α10 nicotinic acetylcholine receptor IC₅₀ value of the RgIA4 peptide, or no greater than 3.0× the α9α10 nicotinic acetylcholine receptor IC₅₀ value of the RgIA4 peptide, or no greater than 5.0× the α9α10 nicotinic acetylcholine receptor IC₅₀ value of the RgIA4 peptide.

In one aspect, the method can comprise providing the α9α10 nicotinic acetylcholine receptor with a selected selectivity. In one example, the RgIA4 peptide analog can provide a α9α10 nicotinic acetylcholine receptor (nAChR) selectivity that is at least one or more of 5×, 10×, 20×, 50×, 100×, or 200×, 500×, or 1000× more selective for the α9α10 nicotinic acetylcholine receptor compared to a selectivity of a different nAChR subtype.

In another aspect, the method can comprise providing a selected stability in human serum compared to an RgIA4 peptide. In one aspect, the RgIA4 peptide analog can provide a stability in human serum of the α-RgIA4 peptide analog of at least one or more of 10%, 20%, 40%, 60%, 80%, 100%, 200%, 300%, 400%, 500%, or 1000% greater than the stability in human serum of an α-RgIA4 peptide.

In another aspect, the method can comprise providing a selected stability in reduced glutathione compared to an RgIA4 peptide. In one example, the RgIA4 peptide analog can provide a stability in reduced glutathione that is at least one or more of 10%, 20%, 40%, 60%, 80%, 100%, 200%, 300%, 400%, 500%, or 1000% greater than the stability of the RgIA4 peptide in the reduced glutathione.

In another aspect, the method can comprise providing a selected solubility in human serum compared to an RgIA4 peptide. In one example, the RgIA4 peptide analog can provide a solubility that is at least one or more of: 5×, 10×, 20×, 50×, 100×, or 200×, 500×, or 1000× greater than the solubility of the RgIA4 peptide analog without the C-terminal residue.

### Compositions and Dosage Forms

With this in mind, in one more embodiment, a composition can include a combination of a therapeutically effective amount of an analog disclosed herein with a pharmaceutically acceptable carrier.

In one aspect, the analog can be present at a concentration of from about 0.0001 wt % to about 10 wt %. In one example, the analog can be present in the composition at a concentration of from about 0.0001 wt % to about 1 wt %. In another example, the analog can be present in the composition at a concentration of from about 0.001 wt % to about 1 wt %. In one more example, the analog can be present in the composition at a concentration of from about 0.01 wt % to about 0.1 wt %. In some examples, the analog can be present in the composition at a concentration of from about 0.005 wt % to about 0.05 wt %.

In one aspect, the pharmaceutically acceptable carrier can include one or more of water, a tonicity agent, a buffering agent, a preservative, the like, or a combination thereof. In some examples, the carrier can include a tonicity agent. Non-limiting examples of tonicity agents can include sodium chloride, potassium chloride, calcium chloride, magnesium chloride, mannitol, sorbitol, dextrose, glycerin, propylene glycol, ethanol, trehalose, phosphate-buffered saline (PBS), Dulbecco's PBS, Alsever's solution, Tris-buffered saline (TBS), water, balanced salt solutions (BSS), such as Hank's BSS, Earle's BSS, Grey's BSS, Puck's BSS, Simm's BSS, Tyrode's BSS, and BSS Plus, the like, or combinations thereof. The tonicity agent can be used to provide an appropriate tonicity of the composition. In one aspect, the tonicity of the composition can be from about 250 to about 350 milliosmoles/liter (mOsm/L). In another aspect, the tonicity of the composition can be from about 277 to about 310 mOsm/L.

In some examples, the carrier can include a pH adjuster or buffering agent. Non-limiting examples of pH adjusters or buffering agents can include a number of acids, bases, and combinations thereof, such as hydrochloric acid, phosphoric acid, citric acid, sodium hydroxide, potassium hydroxide, calcium hydroxide, acetate buffers, citrate buffers, tartrate buffers, phosphate buffers, triethanolamine (TRIS) buffers, the like, or combinations thereof. Typically, the pH of the therapeutic composition can be from about 5 to about 9, or from about 6 to about 8. In another example, the pH of the therapeutic composition can be from about 5 to about 6.

In some examples, the carrier can include a preservative. Non-limiting examples of preservatives can include ascorbic acid, acetylcysteine, bisulfite, metabisulfite, monothioglycerol, phenol, meta-cresol, benzyl alcohol, methyl paraben, propyl paraben, butyl paraben, benzalkonium chloride, benzethonium chloride, butylated hydroxyl toluene, myristyl gamma-picolimium chloride. 2-phenoxyethanol, phenyl mercuric nitrate, chlorobutanol, thimerosal, tocopherols, the like, or combinations thereof.

In one aspect, the composition can further comprise an additional active agent. In one aspect, the additional active agent is a member selected from the group consisting of: an anti-inflammatory agent, an anesthetic, a secondary analgesic peptide, a non-peptide analgesic, the like, or a combination thereof.

In one example, the additional active agent can be an anti-inflammatory agent. Non-limiting examples of anti-inflammatory agents can include ibuprofen, naproxen, aspirin, diclofenac, celecoxib, sulindac, oxaprozin, piroxicam, indomethacin, meloxicam, fenoprofen, difunisal, etodolac, ketorolac, meclofenamate, nabumetone, salsalate, ketoprofen, tolmetin, flurbiprofen, mefenamic acid, famotidine, bromfenac, nepafenac, prednisone, cortisone, hydrocortisone, methylprednisolone, deflazacort, prednisolone, fludrocortisone, amcinonide, betamethasone diproprionate, clobetasol, clocortolone, dexamethasone, diflorasone, durasteride, flumethasone pivalate, flunisolide, fluocinolone acetonide, fluocinonide, fluorometholone, fluticasone propionate, flurandrenolide, hydroflumethiazide, the like, hydrates thereof, acids thereof, bases thereof, or salts thereof. or combinations thereof.

In one example, the additional active agent can be an anesthetic. Non-limiting examples of anesthetics can include articaine, bupivacaine, cinchocaine, etidocaine, levobupivacaine, lidocaine, mepivacaine, prilocaine, ropivacaine, trimecaine, the like, or combinations thereof.

In one example, the additional active agent can be a secondary analgesic peptide. In one example, the additional active agent can be a non-peptide analgesic. Non-limiting examples of non-peptide analgesics can include acetaminophen, codeine, dihydrocodeine, tramadol, meperidine, hydrocodone, oxycodone, morphine, fentanyl, hydromorphone, buprenorphine, methadone, diamorphine, pethidine, the like, hydrates thereof, acids thereof, bases thereof, or salts thereof, or combinations thereof.

In one aspect, the additional active agent can be present at a concentration of from about 0.0001 wt % to about 10 wt %. In one example, the additional active agent can be present in the composition at a concentration of from about 0.0001 wt % to about 1 wt %. In another example, the additional active agent can be present in the composition at a concentration of from about 0.001 wt % to about 1 wt %. In one more example, the additional active agent can be present in the composition at a concentration of from about 0.01 wt % to about 0.1 wt %. In some examples, the additional active agent can be present in the composition at a concentration of from about 0.005 wt % to about 0.05 wt %.

In another aspect, the composition can be formulated as one of: a solution, a suspension, an emulsion, a gel, a hydrogel, a thermo-responsive gel, a cream, an ointment, a paste, an adhesive, a liquid reservoir, a patch, or a combination thereof. In some aspects, the composition can be suitable for topical, transdermal, intravenous, subcutaneous administration, the like, or a combination thereof. In one aspect, the composition can be suitable for subcutaneous injection.

### Methods of Treatment

In yet another embodiment, a method for treating in a subject, a condition that is responsive to α9α10 nicotinic acetylcholine receptor binding can include administering a therapeutically effective amount of the composition to the subject. In one aspect, the condition can be pain. In another aspect, the condition can be spinal polyradiculopathy. In another aspect, the condition can be postherpetic neuralgia. In another aspect, the condition can be trigeminal neuralgia. In another aspect, the condition can be complex regional pain syndrome. In another aspect, the condition can be multiple sclerosis.

When the condition is pain, the pain can be neuropathic pain including one or more of: chemo-induced neuropathy (CIPN), diabetic neuropathy, arthritic neuropathy, osteoarthritic neuropathy, the like, or a combination thereof. In another aspect, the pain can be HIV pain. In another aspect, the pain can be pain associated with leprosy. In another aspect, the pain can be one or more of post-surgical pain, post-traumatic pain, the like, or a combination thereof.

In another aspect, the condition can be cancer. The cancer can include one or more of: epithelial cancer, lung cancer, breast cancer, the like, or a combination thereof.

In another aspect, the condition can be inflammation. In one aspect, the inflammation can be mediated by immune cells, associated with rheumatism, the like, or a combination thereof. Illustrative inflammatory conditions that can be treated include inflammation, chronic inflammation, rheumatic diseases (including arthritis, lupus, ankylosing spondylitis, fibromyalgia, tendonitis, bursitis, scleroderma, and gout), sepsis, fibromyalgia, inflammatory bowel disease (including ulcerative colitis and Crohn's disease), sarcoidosis, endometriosis, uterine fibroids, inflammatory skin diseases (including psoriasis and impaired wound healing), inflammatory conditions of the lungs (including asthma and chronic obstructive pulmonary disease), diseases associated with inflammation of the nervous system (including multiple sclerosis, Parkinson's disease and Alzheimer's disease), periodontal disease, and cardiovascular disease.

In one aspect, the composition can be a dosage form having from about 25 μl to about 1 ml of the α-RgIA4 analog. In another aspect, the composition can be a dosage form having from about 1 ml to about 5 ml of the α-RgIA4 analog. In one aspect, the composition can be a dosage form having from about 5 ml to about 10 ml of the α-RgIA4 analog.

In another embodiment, treatment can provide a reduction in symptoms within a selected amount of time after administration. Administering the therapeutically effective amount of the topical composition can reduce the symptoms associated with the condition. In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In yet another aspect, the therapeutically effective amount of the composition can be administered to the subject 1 to 10 times per day. In one example, the composition can be administered to the subject 1 to 10 times per day. In another example, the composition can be administered to the subject 1 to 5 times per day. In yet another example, the composition can be administered to the subject 3 to 5 times per day.

In one more aspect, the therapeutically effective amount of the composition can be administered to the subject according to a dosage regimen. In one example, the composition can be administered at least once per day for a duration of from about a single day to about 12 months. In another example, the composition can be administered at least once per day for a duration of from about a single day to about 6 months. In one more example, the composition can be administered at least once per day for a duration of from about a single day to about 3 months. In yet another example, the composition can be administered at least once per day for a duration of from about a single day to about 1 month.

In another aspect, administering the therapeutically effective amount of the composition can be as a subcutaneous dosage form, a transdermal dosage form, a topical dosage form, an intravenous dosage form, the like, or a combination thereof.

In another aspect, a composition for use in the treatment of a condition in a subject that is responsive to α9α10 nicotinic acetylcholine receptor binding can comprise a therapeutically effective amount of the composition. In another aspect, the use of a composition in the manufacture of a medicament for the treatment of a condition in a subject that is responsive to α9α10 nicotinic acetylcholine receptor binding can comprise a therapeutically effective amount of the composition.

### Sequence Listing

Table 1 sets forth the sequences for RgIA, RgIA4 and RgIA4 analogues.

TABLE 1

| | | SEQ |
|---|---|---|
| Pep- | | ID |
| tide | SEQUENCE | NO. |
| RgIA | G C C S D P R C R Y R C R | 1 |
| RgIA4 | G C C T D P R C (Cit) (iY) Q C Y | 2 |
| | G C X1 T D P R C X2 (R-3-Y) Q C | 3 |
| | X3 X4 | |
| | G C L-Pen T D P R C R (R-3-Y) Q | 4 |
| | C X3 X4 | |
| | G C L-Pen T D P R C R (I-3-Y) Q | 5 |
| | C X3 X4 | |
| | G C L-Pen T D P R C R (I-3-Y) Q | 6 |
| | C (bhY) X4 | |
| | G C L-Pen T D P R C R (I-3-Y) Q | 7 |
| | C (3-R-bhY) X4 | |
| | G C L-Pen T D P R C R (I-3-Y) Q | 8 |
| | C (bhY) R | |
| | G C L-Pen T D P R C R (I-3-Y) Q | 9 |
| | C (3-R-bhY) R | |
| | G C L-Pen T D P R C X5 (I-3-Y) Q | 10 |
| | C β³hY X6 | |
| | G C (Pen) T D P R C X5 ^{I3}Y Q C | 11 |
| | 3-R-β³hY X6 | |
| RgIA-5474 | G C L-Pen T D P R C R (I-3-Y) Q | 12 |
| | C β³hY R | |
| RgIA-5439 | G L-Pen C T D P R C (Cit) (I-3-Y) | 13 |
| | Q C Y | |
| RgIA-5408 | G C L-Pen T D P R C (Cit) (I-3-Y) | 14 |
| | Q C Y | |
| RgIA-5440 | G C C T D P R L-Pen (Cit) (I-3-Y) | 15 |
| | QCY | |
| RgIA-5409 | G C C T D P R C (Cit) (I-3-Y) Q | 16 |
| | L-Pen Y | |
| RgIA-5493 | G L-Pen C T D P R L-Pen (Cit) | 17 |
| | (I-3-Y) Q C Y | |
| RgIA-5446 | G C L-Pen T D P R C (Cit) (I-3-Y) | 18 |
| | Q L-Pen Y | |
| RgIA-5432 | G C L-Pen T D P R C R (I-3-Y) Q C | 19 |
| | Y | |
| RgIA-5624 | G C L-Pen T D P R C R (I-3-Y) Q C | 20 |
| | Y R | |
| RgIA-5433 | G C L-Pen T D P R C R (I-3-Y) Q C | 21 |
| | y r | |
| RgIA-5477 | G C L-Pen T D P R C R (I-3-Y) Q C | 22 |
| | β³hY r | |
| RgIA-5702 | G C L-Pen T D P R C R (I-3-Y) Q C | 23 |
| | β³hY K | |
| RgIA-5672 | G C L-Pen T D P R C R (I-3-Y) Q C | 24 |
| | β³hY Orn | |
| RgIA-5686 | G C L-Pen T D P R C R (I-3-Y) Q C | 25 |
| | β³hY Cit | |
| RgIA-5687 | G C L-Pen T D P R C R (I-3-Y) Q C | 26 |
| | β³hY E | |
| RgIA-5711 | G C C T D P R C R (I-3-Y) Q C β³hY | 27 |
| | R | |

### EXAMPLES

In one example, a synthetic analgesic peptide can comprise the amino acid sequence GCX1 TD P R C X2 (R-3-Y) QC X3 X4 (SEQ ID NO: 3), wherein X1 is selected from the group consisting of L-penicillamine (L-Pen), D-penicillamine (D-Pen), and L-cysteine, X2 is selected from the group consisting of L-arginine, D-arginine, or citrulline, X3 is any amino acid, X4 is any amino acid, and R-3-Y is 3-R-tyrosine, wherein 3-R-tyrosine can be a peptide residue selected from the group consisting of 3-chloro-tyrosine, 3-fluoro-tyrosine, 3-iodo-tyrosine, 3-bromo-tyrosine, and tyrosine. In another example, X1 can be L-Pen.

In another example, X2 can be L-arginine.

In another example, X3 can be selected from the group consisting of beta-homo-tyrosine, L-tyrosine, and D-tyrosine.

In another example, X4 can be selected from the group consisting of L-arginine and D-arginine.

In another example, the synthetic analgesic peptide can comprise the amino acid sequence G C L-Pen T D P R C R (R-3-Y) Q C X3 X4 (SEQ ID NO: 4).

In another example, the synthetic analgesic peptide can comprise the amino acid sequence G C L-Pen T D P R CR (1-3-Y) QC X3 X4 (SEQ ID NO: 5).

In another example, the synthetic analgesic peptide can comprise the amino acid sequence G C L-Pen T D P R C R (1-3-Y) Q C (bhY) X4 (SEQ ID NO: 6).

In another example, the synthetic analgesic peptide can comprise the amino acid sequence G C L-Pen T D P R CR (1-3-Y) Q C (3-R-bhY) X4 (SEQ ID NO: 7).

In another example, the synthetic analgesic peptide can comprise the amino acid sequence G C L-Pen T D P R C R (I-3-Y) Q C (bhY) R (SEQ ID NO: 8).

In another example, the synthetic analgesic peptide can comprise the amino acid sequence G C L-Pen T D P R C R (I-3-Y) Q C (3-R-bhY) R (SEQ ID NO: 9).

In another example, a method for treating or preventing a condition or disorder associate with α9α10 subtype of the nicotinic acetylcholine receptor (nAChR) in a subject can comprise administering to the subject a therapeutically effective amount of a composition comprising the synthetic analgesic peptide.

In another example, a method for reducing pain in a subject can comprise administering to the subject a therapeutically effective amount of a composition comprising the synthetic analgesic peptide,

In another example, a method for reducing or alleviating pain associated with diabetic neuropathy pain in a subject, can comprise administering to the subject a therapeutically effective amount of a composition comprising the synthetic analgesic peptide.

In another example, a method for reducing or alleviating chemotherapy-induced pain in a subject, can comprise administering to the subject a therapeutically effective amount of a composition comprising the synthetic analgesic peptide.

In another example, a method for increasing a stability of a synthetic analgesic peptide, can comprise providing the synthetic analgesic peptide.

In another example, a method for increasing a potency of a synthetic analgesic peptide, can comprise providing the synthetic analgesic peptide.

In another example, a synthetic analgesic peptide can comprise an amino acid sequence selected from the group consisting of SEQ ID NO: 3 through SEQ ID NO: 9.

In another example, an RgIA4 peptide analog can comprise a recognition finger region configured to bind to an α9α10 nicotinic acetylcholine receptor: and at least two disulfide bridges comprising: an inter-cysteine disulfide bridge between a first cysteine and a second cysteine, and a penicillamine-cysteine disulfide bridge between a first penicillamine and a third cysteine: wherein the RgIA4 peptide analog has a binding affinity for the α9α10 nicotinic acetylcholine receptor that is at least 50% of a binding affinity of an RgIA4 peptide.

In another example, the RgIA4 peptide analog can comprise a β³-homo tyrosine bound to the third cysteine.

In another example, the binding affinity for the α9α10 nicotinic acetylcholine receptor can be: substantially equal to the binding affinity of the RgIA4 peptide, or greater than the binding affinity of an RgIA4 peptide.

In another example, the penicillamine-cysteine disulfide bridge can provide an increase in potency compared to a potency of an RgIA4 peptide.

In another example, the RgIA4 peptide analog can provide an α9α10 nicotinic acetylcholine receptor IC₅₀ value that is: at least 25.0× less than the α9α10 nicotinic acetylcholine receptor IC₅₀ value of the RgIA4 peptide, or at least 10.0× less than the α9α10 nicotinic acetylcholine receptor IC₅₀ value of the RgIA4 peptide, or at least 5.0× less than the α9α10 nicotinic acetylcholine receptor IC₅₀ value of the RgIA4 peptide, or at least 2.0× less than the α9α10 nicotinic acetylcholine receptor IC₅₀ value of the RgIA4peptide, or substantially equal to an α9α10 nicotinic acetylcholine receptor IC₅₀ value of the RgIA4 peptide, or no greater than 2.0× the α9α10 nicotinic acetylcholine receptor IC₅₀ value of the RgIA4 peptide, or no greater than 3.0× the α9α10 nicotinic acetylcholine receptor IC₅₀ value of the RgIA4 peptide, or no greater than 5.0× the α9α10 nicotinic acetylcholine receptor IC₅₀ value of the RgIA4 peptide.

In another example, the penicillamine-cysteine disulfide bridge can reduce one or more of disulfide bridge scrambling, disulfide bridge degradation, or a combination thereof as compared to: an RgIA4 peptide, or an RgIA4 peptide analog without a penicillamine-cysteine disulfide bridge.

In another example, the penicillamine-cysteine disulfide bridge can provide a stability for the RgIA4 peptide analog in human serum that is greater than the stability of an RgIA4 peptide in human serum, wherein the stability in the human serum is measured by the amount of a globular form of the RgIA4 peptide analog remaining after incubation of the RgIA4 peptide analog in 25% human serum AB type and incubated at 37° C. for at least one of 1, 2, 4, 8, 24, 48, or 72 hours.

In another example, the stability in human serum of the RgIA4 peptide analog can be at least one or more of 10%, 20%, 40%, 60%, 80%, 100%, 200%, 300%, 400%, 500%, or 1000% greater than the stability of the RgIA4 peptide in human serum.

In another example, the RgIA4 peptide analog can provide an α9α10 nicotinic acetylcholine receptor selectivity that is substantially equal to the α9α10 nicotinic acetylcholine receptor selectivity of an RgIA4 peptide.

In another example, the RgIA4 peptide analog can provides a α9α10 nicotinic acetylcholine receptor selectivity that is at least one or more of 5×, 10×, 20×, 50×, 100×, or 200×, 500×, or 1,000× more selective for the α9α10 nicotinic acetylcholine receptor compared to a selectivity of a different nicotinic acetylcholine receptor (nAChR) subtype.

In another example, the different nAChR subtype can be selected from the group consisting of: α1β1δϵ, α2β2, α2β4, α3β2, α3β4α4β2, α4β4, αβ/α3β2β3, αβ/α3β4, α7, or a combination thereof.

In another example, the RgIA4 peptide analog can comprise a C-terminal residue selected from the group consisting of L-citrulline, D-citrulline, L-arginine, D-arginine, L-lysine, D-lysine, L-ornithine, and D-ornithine.

In another example, the RgIA4 peptide analog can provide a solubility that is at least one or more of: 5×, 10×, 20×, 50×, 100×, or 200×, 500×, or 1000× greater than the solubility of the RgIA4 peptide analog without the C-terminal residue.

In another example, the RgIA4 peptide analog can provide a stability in reduced glutathione that is greater than the stability of an RgIA4 peptide in reduced glutathione, wherein the stability in the reduced glutathione is measured by the amount remaining after incubation of 0.1 mg/mL of the RgIA4 peptide analog or the RgIA4 peptide in 10 equivalents of reduced glutathione in phosphate buffered saline (PBS) having a pH of 7.4 and incubated at 37° C. for at least one of 1, 2, 4, 8, 24, 48, or 72 hours.

In another example, the stability in the reduced glutathione of the α-RgIA4 peptide analog can be at least one or more of 10%, 20%, 40%, 60%, 80%, 100%, 200%, 300%, 400%, 500%, or 1000% greater than the stability of the RgIA4 peptide in the reduced glutathione.

In another example, the RgIA4 peptide analog can provide a safety profile that is substantially equal to or greater than the safety profile of an RgIA4 peptide, wherein the safety profile is measured by one or more of: the analog present in a concentration of 100 μM inhibits less than 25% of the human ether-a-go-go-related gene (hERG) K+ channel as measured from an automated-whole cell patch-clamp assay, or the analog present in a concentration of 10 μM has inhibitory activity of less than 20% as measured in a CYP assay.

In another example, the RgIA4 peptide analog can comprise the amino acid sequence GC (Pen) T D P R C X5 13Y Q C β³hY X6 (SEQ ID NO: 10), wherein: X5 is selected from the group consisting of L-citrulline, D-citrulline, L-arginine or D-arginine, and X6 is selected from the group consisting of L-arginine or D-arginine.

In another example, X5 can be L-arginine and X6 can be L-arginine.

In another example, the RgIA4 peptide analog can comprise the amino acid sequence G C (Pen) T D PR C X5 13Y Q C (3-R-β³hY) X6 (SEQ ID NO: 11), wherein: X5 is selected from the group consisting of L-citrulline, D-citrulline, L-arginine or D-arginine, and X6 is selected from the group consisting of L-arginine or D-arginine.

In another example, X5 can be L-arginine and X6 can be L-arginine.

In another example, a composition can comprise a combination of a therapeutically effective amount of an RgIA4 peptide analog with a pharmaceutically acceptable carrier.

In another example, the composition can be suitable for topical, transdermal, intravenous, or subcutaneous administration.

In another example, the composition can further comprise an additional active agent.

In another example, the additional active agent can be a member selected from the group consisting of: an anti-inflammatory agent, an anesthetic, a secondary analgesic peptide, a non-peptide analgesic, and combinations thereof.

In another example, the additional active agent can be present at a concentration of from about 0.0001 wt % to about 10 wt %.

In another example, the composition can be formulated as one of: a solution, a suspension, an emulsion, a gel, a hydrogel, a thermo-responsive gel, a cream, an ointment, a paste, an adhesive, a liquid reservoir, a patch, or a combination thereof.

In another example, the composition can be suitable for subcutaneous injection.

In another example, the pharmaceutically acceptable carrier can include one or more of water, a tonicity agent, a buffering agent, a preservative, or a combination thereof.

In another example, a method of maintaining an RgIA4 peptide potency for an α9α10 nicotinic acetylcholine receptor in an RgIA4 peptide analog can comprise: providing an inter-cysteine disulfide bridge between a first cysteine and a second cysteine, and a penicillamine-cysteine disulfide bridge between a first penicillamine and a third cysteine: providing a β³-homo tyrosine bound to the third cysteine: and providing a C-terminal residue selected from the group consisting of L-citrulline, D-citrulline, L-arginine, D-arginine, L-lysine, D-lysine, L-ornithine, and D-ornithine.

In another example, the RgIA4 peptide analog can inhibit the α9α10 nicotinic acetylcholine receptor with an IC₅₀ value that is: at least 25.0× less than the α9α10 nicotinic acetylcholine receptor IC₅₀ value of the RgIA4 peptide, or at least 10.0× less than the α9α10 nicotinic acetylcholine receptor IC₅₀ value of the RgIA4 peptide, or at least 5.0× less than the α9α10 nicotinic acetylcholine receptor IC₅₀ value of the RgIA4 peptide, or at least 2.0x less than the α9α10 nicotinic acetylcholine receptor IC₅₀ value of the RgIA4peptide, or substantially equal to an α9α10 nicotinic acetylcholine receptor IC₅₀ value of the RgIA4 peptide, or no greater than 2.0× the α9α10 nicotinic acetylcholine receptor IC₅₀ value of the RgIA4 peptide, or no greater than 3.0× the α9α10 nicotinic acetylcholine receptor IC₅₀ value of the RgIA4 peptide, or no greater than 5.0× the α9α10 nicotinic acetylcholine receptor IC₅₀ value of the RgIA4 peptide.

In another example, the RgIA4 peptide analog can provide a α9α10 nicotinic acetylcholine receptor (nAChR) selectivity that is at least one or more of 5×, 10×, 20×, 50×, 100×, or 200×, 500×, or 1000× more selective for the α9α10 nicotinic acetylcholine receptor compared to a selectivity of a different nAChR subtype.

In another example, the RgIA4 peptide analog can provide a stability in human serum of the α-RgIA4 peptide analog of at least one or more of 10%, 20%, 40%, 60%, 80%, 100%, 200%, 300%, 400%, 500%, or 1000% greater than the stability in human serum of an α-RgIA4 peptide.

In another example, the RgIA4 peptide analog can provide a solubility that is at least one or more of: 5×, 10×, 20×, 50×, 100×, or 200×, 500×, or 1000× greater than the solubility of the RgIA4 peptide analog without the C-terminal residue.

In another example, the RgIA4 peptide analog can provide a stability in reduced glutathione that is at least one or more of 10%, 20%, 40%, 60%, 80%, 100%, 200%, 300%, 400%, 500%, or 1000% greater than the stability of the RgIA4 peptide in the reduced glutathione.

In another example, a method for treating in a subject, a condition that is responsive to α9α10 nicotinic acetylcholine receptor binding can comprise administering a therapeutically effective amount of the composition to the subject.

In another example, the condition can be pain.

In another example, the pain can be neuropathic pain including one or more of: chemo-induced neuropathy (CIPN), diabetic neuropathy, arthritic neuropathy, osteoarthritic neuropathy, or a combination thereof.

In another example, the pain can be HIV pain.

In another example, the pain can be pain associated with leprosy.

In another example, the pain can be one or more of post-surgical pain, or post-traumatic pain.

In another example, the condition can be spinal polyradiculopathy.

In another example, the condition can be postherpetic neuralgia.

In another example, the condition can be trigeminal neuralgia.

In another example, the condition can be complex regional pain syndrome.

In another example, the condition can be cancer.

In another example, the cancer can include one or more of: epithelial cancer, lung cancer, breast cancer, or a combination thereof.

In another example, the condition can be multiple sclerosis.

In another example, the condition can be inflammation.

In another example, the inflammation can be mediated by immune cells, associated with rheumatism, or a combination thereof.

In another example, the treatment can provide a reduction in symptoms of at least 10% within a selected amount of time after administration.

In another example, the method can comprise administering the therapeutically effective amount of the composition to the subject 1 to 5 times per day.

In another example, the method can comprise administering the therapeutically effective amount of the composition to the subject according to a dosage regimen of at least once per day for a duration of from about a single day to about 3 months.

In another example, the method can comprise administering the therapeutically effective amount of the composition as a subcutaneous dosage form, a transdermal dosage form, a topical dosage form, an intravenous dosage form, or a combination thereof.

In another example, a composition for use in the treatment of a condition in a subject that is responsive to α9α10 nicotinic acetylcholine receptor binding can comprise a therapeutically effective amount of the composition to the subject.

In another example, use of a composition in the manufacture of a medicament for the treatment of a condition in a subject that is responsive to α9α10 nicotinic acetylcholine receptor binding can comprise a therapeutically effective amount of the composition to the subject.

### EXPERIMENTAL EXAMPLES

The following examples are provided to promote a more clear understanding of certain embodiments of the present disclosure, and are in no way meant as a limitation thereon.

### Example 1

### Solid Phase Peptide Synthesis

All peptides described herein were synthesized on 0.05 mmol scale using an Apex 396 automated peptide synthesizer (AAPPTec: Louisville, KY), applying standard solid-phase Fmoc (9-fluorenylmethyloxycarbonyl) protocols using Fmoc-Tyr(tBu) Wang resin (0.49 mmol/g load: Peptides International, Louisville, KY), Fmoc-Arg(Pbf)-Wang resin (0.3 mmol/g load: Peptides International), Fmoc-Lys(Boc)-Wang resin (0.3 mmol/g load: Peptides International), Fmoc-Orn(Boc)-Wang resin (0.73 mmol/g: Bachem, Torrance, CA), Fmoc-Cit-Wang resin (0.42 mmol/g: Santa Cruz Biotechnology, Dal-las, TX), Fmoc-Glu(OtBu)-Wang resin LL (Millipore Sigma, Burlington, MA) and Fmoc-Asp(OtBu)-Wang resin (0.51 mmol/g, Peptides International).

All standard amino acids, Fmoc-Pen(Trt)-OH (Pen), Fmoc-Cit-OH and Fmoc-beta-HTyr(tBu)-OH were purchased from AAPPTec except for Fmoc-3-iodo-L-Tyr-OH (Peptides International)(I3Y). Side-chain protection for the following amino acids was as follows: Arg. 2,2,4,6,7-pentamethyl-dihydrobenzofuran-5-sulfonyl (Pbf): Thr, Tyr, beta-HTyr, tert-butyl (tBu): Lys, tert-butyloxycarbonyl (Boc): Gln, trityl (Trt). To promote the correct folding of the disulfide bridge a trityl protected Pen residue was paired either with another Pen (Trt) or with trityl protected Cys residue, while the other pair of Cys residues were acetamidomethyl (Acm) protected. Coupling activation was achieved with 1 eq of 0.4 M benzotriazol-1-yloxytripyrrolidino-phosphonium hexafluorophosphate (ChemImpex: Wood Dale, IL) and 2 eq of 2 M N,N-diisopropylethyl amine (Millipore Sigma, St. Louis, MO) in N-methyl-2-pyrrolidone (Fisher Scientific: Waltham, MA) as the solvent. For each coupling reaction, a 10-fold excess of the standard amino acid and 5-fold excess of the special amino acid was used, and the reaction was carried out for 60 and 90 min respectively. Fmoc deprotection was per-formed for 20 min with 20% (v/v) piperidine (Alfa Aesar: Tewksbury, MA) in N,N-dimethylformamide (Fisher Scientific).

The use of non-natural amino acids in-creases the cost of materials for synthesis of linear peptide. For a 0.05 mmol scale synthesis, the approximate cost per residue, based on the price of the amino acids and resins, is $1.2 for RgIA, $2.9 for RgIA4 and RgIA5 and $9 for RgIA-5474.

### Example 2

### Peptide Cleavage, Purification, and Oxidative Folding

Peptide Cleavage. Purification and Oxidative Folding

The protocols described for RgIA-5474 cleavage from the resin, purification, and two-operation oxidative folding were used for the synthesized peptides describe herein.

### RgIA-5474 Cleavage and Purification

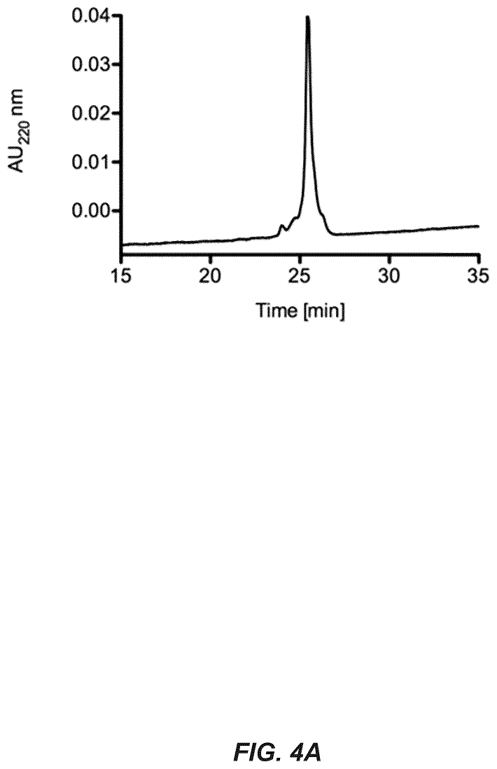

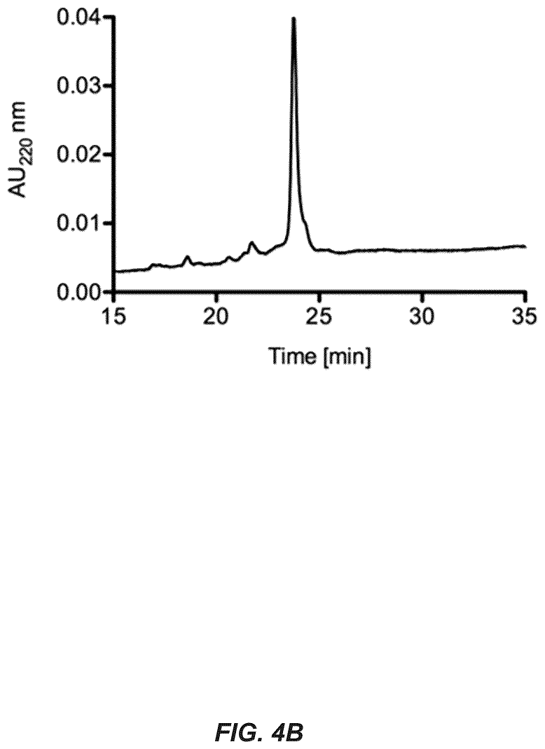

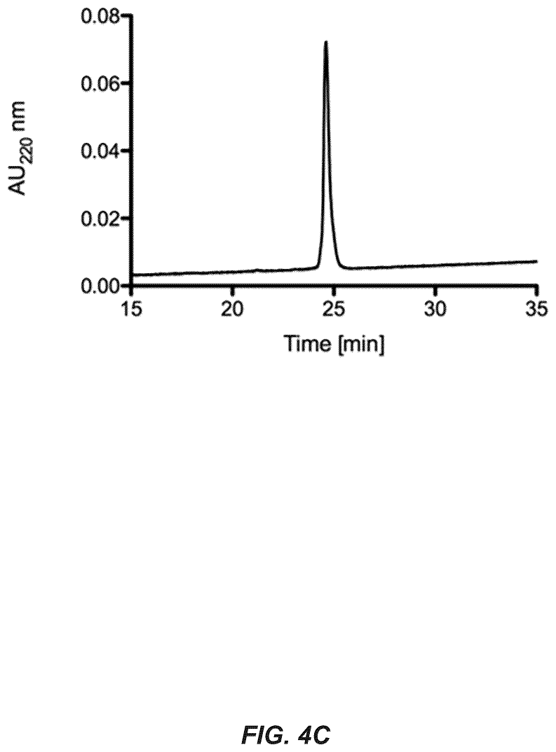

The peptides were cleaved from the resin using Reagent K consisting of tri-fluoroacetic acid (TFA)/phenol/ethanedithiol/thioanisol/H₂O (9:0.75:0.25: 0.5:0.5 by volume) (Fisher Scientific, Millipore Sigma, Millipore Sigma and Acros Organics respectively). Next, the cleavage mixture was filtered and precipitated with 150 mL of cold methyl-tert-butyl ether (MTBE: Fisher Scientific). The crude peptide was then precipitated by centrifugation at 7,000× g for 7 min and washed twice with 150 mL cold MTBE. The crude peptide was diluted with 50 mL of the HPLC buffer B and purified by reverse-phase (RP) HPLC using a preparative C18 Vydac column (218TP101522, 250×22 mm, 5-μm particle size) eluted with a linear gradient ranging from 10% to 50% buffer B in 40 min at a flow rate 20 mL/min. The HPLC buffers were 0.1% (vol/vol) TFA in water (buffer A) and 0.092% TFA (vol/vol) in 60% aqueous acetonitrile (Fisher Scientific) (vol/vol) (buffer B). The eluent was monitored by measuring absorbance at 220/280 nm. Purity of the peptide was assessed by analytical C18 Vydac RP-HPLC (218TP54, 250×4.6 mm, 5-μm particle size) using the same gradient as described above with a flow rate 1 mL/min. From ˜200 mg resin cleaved 4,318 nmols of linear RgIA-5474 was prepared. The HPLC trace of the linear RgIA-5474 is provided in FIG. 4A. The HPLC trace was collected by RP-HPLC using C18 column and a linear gradient ranging from 10% to 50% buffer B in 40 min.

### Example 3

### Disulfide Bond Formation

### RgIA-5474 First Disulfide Bond Formation (2Cys-8Cys).

In a solution consisting of 20 mM potassium ferricyanide (0.659 g: 2 mmol: Millipore Sigma) and 0.1M Tris base (1.21 g: 10 mmol: Millipore Sigma), dissolved in 100 mL of nano-pure water, 4,318 nmols of linear RgIA-5474, diluted with buffer A to total volume of 150 mL, was added drop-wise (peptide final concentration was approximately 20) μM and the pH was 7.5). The reaction was carried out for 45 min at room temperature, then terminated by diluting it with 250 mL buffer A to drop the pH. The reaction mixture was then passed through a disposable C18 cartridge (Thermo Fisher, Hypersep Spe C18 1000 mg/8 mL), and the peptide was eluted using buffer B. The efficiency of the reaction as well as the purity of the peptide was analyzed by analytical RP-HPLC as described for the linear peptide. From 4.318 nmols of the linear peptide, of 2,387 nmols of monocyclic RgIA-5474 was obtained 55% yield, 85% purity). The HPLC trace of the monocyclic RgIA-5474 is provided in FIG. 4B. The HPLC trace was collected by RP-HPLC using C18 column and a linear gradient ranging from 10% to 50% buffer B in 40 min.

### RgIA-5474 Second Disulfide Bond Formation (3Pen-12Cys).

Removal of the acetamidomethyl groups and the second disulfide bridge formation was accomplished by iodine oxidation. 76 mg of iodine (2 mmols: Acros Organics) was add-ed to 15 mL of acetonitrile and stirred until completely dis-solved. Then, 45 mL of nanopure water was added followed by 1.8 mL of TFA. The monocyclic RgIA-5474 solution of 2,387 nmols diluted with 90 mL buffer A, was dripped into 60 mL of the 10 mM iodine solution (prepared as described above) and allowed to react for 10 min at room temperature. Peptide final concentration was kept close to 20 μM. The reaction was quenched by adding 5-10 drops of 1 M freshly prepared ascorbic acid (0.176 g, 1 mmol: Research Products International, Mount Prospect. IL) solution in water (1 mL) until the reaction mixture became transparent. The reaction was then diluted 5-fold with buffer A and subsequently purified by RP-HPLC using a preparative C18-column as described for the linear peptide to obtain 1,568 nmols of the fully folded RgIA-5474.

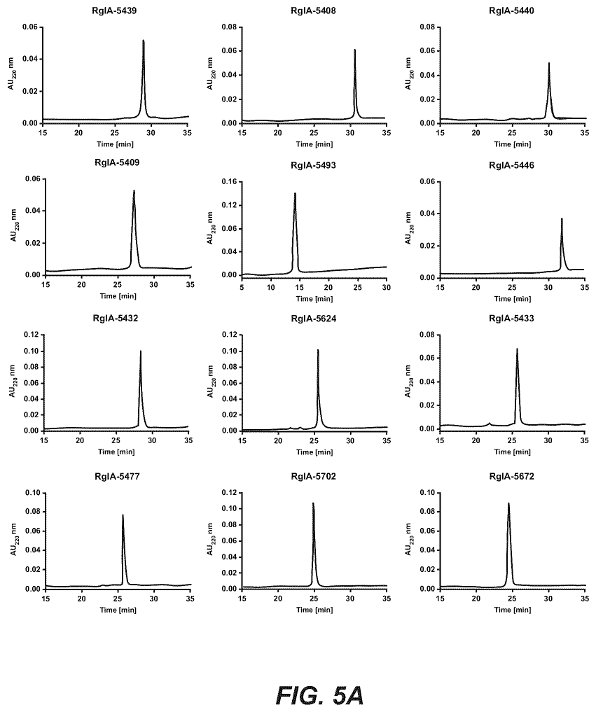

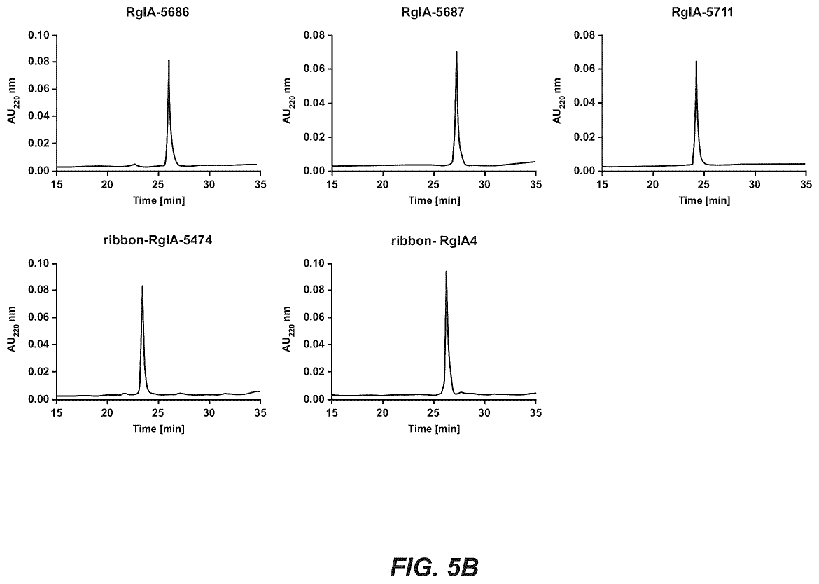

Purity and final yield of RgIA-5474 was determined by RP-HPLC using analytical C18 column using the gradients described earlier for linear peptides and was determined to be 96% and 36% (based on the starting amount of the linear peptide) respectively. The calculated mass of RgIA-5474 [MH]+1=1887.75 was verified to be [M+H]+1=1887.59 by matrix-assisted laser desorption ionization time-of-flight (MALDI-TOF) mass spectrometry or electrospray ionization (ESI) at the Mass Spectrometry and Proteomics Core Facility at the University of Utah. The HPLC trace of the fully folded RgIA-5474 is provided in FIG. 4C. The HPLC trace was collected by RP-HPLC using a C18 Vydac RP-HPLC (218TP54, 250×4.6 mm, 5-μm particle size) column and a linear gradient ranging from 10% to 50% buffer B in 40 min with a flow rte of 1 mL/min. The purity for all for all fully folded peptides was >95% and was determined using analytical RP-HPLC, as shown in FIGS. 5A and 5B. Mass Spec results, purity, and retention times (RT) for all fully folded peptides are provided in Table 3A.

TABLE 3A
PURITY, RETENTION TIME, AND MASS SPECTROMETRY
DATA FOR ALL PEPTIDE ANALOGS.

| | Purity | RT | Mass [MH]⁺¹ [Da] | |
|---|---|---|---|---|
| Analog | [%] | [min] | calculated | determined |
| RgIA-5439 | 97 | 28.9 | 1718.49 | 1718.44 |
| RgIA-5408 | 96 | 30.6 | 1718.49 | 1718.50 |
| RgIA-5440 | 99 | 30.0 | 1718.49 | 1718.50 |
| RgIA-5409 | 99 | 27.2 | 1718.49 | 1718.47 |
| RgIA-5493* | 99 | 31.5 | 1746.53 | 1746.55 |
| RgIA-5446 | 99 | 31.8 | 1746.53 | 1746.55 |
| RgIA-5432 | 97 | 28.5 | 1717.50 | 1717.53 |
| RgIA-5624 | 95 | 25.5 | 1873.60 | 1873.58 |
| RgIA-5433 | 96 | 25.6 | 1873.60 | 1873.59 |
| RgIA-5477 | 99 | 25.7 | 1887.75 | 1887.59 |
| RgIA-5702 | 99 | 24.9 | 1859.75 | 1859.65 |
| RgIA-5672 | 99 | 24.4 | 1845.75 | 1845.57 |
| RgIA-5686 | 99 | 26.1 | 1888.75 | 1888.58 |
| RgIA-5687 | 99 | 27.3 | 1860.69 | 1860.53 |
| RgIA-5711 | 96 | 24.2 | 1859.71 | 1859.56 |
| ribbon-RgIA-5474 | 99 | 23.4 | 1887.75 | 1887.60 |
| ribbon-RgIA4 | 95 | 26.3 | 1690.46 | 1690.42 |

For peptides marked with (*) the HPLC traces were rerecorded on a different day and using the same conditions, but different HPLC instrument. RT is retention time.

As illustrated in FIG. 5A, traces were collected at room temperature using analytical C18 Vydac RP-HPLC (218TP54, 250×4.6 mm, 5-μm particle size) and a gradient ranging from 10% B to 50% B in 40 min with a flow rate 1 mL/min using the following buffers: 0.1% (vol/vol) TFA in water (buffer A) and 0.092% TFA (vol/vol) in 60% aqueous acetonitrile (vol/vol) (buffer B). HPLC trace for RgIA-5493 was collected at 45° C. with the gradient ranging 10% B to 50% B in 40 min with a flow rate 1 mL/min with buffer B being 90% acetonitrile in aqueous 0.1% TFA.

As illustrated in FIG. 5B, traces were collected using analytical C18 Vydac RP-HPLC (218TP54, 250×4.6 mm, 5-um particle size) and a gradient ranging from 10% B to 50% B in 40 min with a flow rate 1 mL/min using the following buffers: 0.1% (vol/vol) TFA in water (buffer A) and 0.092% TFA (vol/vol) in 60% aqueous acetonitrile (vol/vol) (buffer B).

### Example 4

### Effect of Selective Penicillamine Substitution on IC₅₀

The approach herein involved the use of penicillamine (Pen). β,β-dimethyl substituted cysteine can introduce small, but favorable, local spatial constraints on the disulfide bridge and the overall peptide conformation. An RgIA4 analog of RgIA can be used. Each Cys residue in the RgIA4 sequence was sequentially substituted with an L-Pen residue and tested on the human α9α10 nAChRs, as displayed in Table 4A.

TABLE 4A
L-Cys to L-Pen substitution in RgIA4 and their
activity on human α9α10 nAChRs.

| | | IC₅₀ | 95% CI |
|---|---|---|---|
| Analog | Sequence | (nM)ª | (nM) |
| RgIA- | G(Pen)CTDPRC(Cit)^{I3}YQCY | >1200 | 702-2200 |
| 5439 | | | |
| RgIA- | GC(Pen)TDPRC(Cit)^{I3}YQCY | 1.3 | 1.03-1.6 |
| 5408 | | | |
| RgIA- | GCCTDPR(Pen)(Cit)^{I3}YQCY | >1000 | ND |
| 5440 | | | |
| RgIA- | GCCTDPRC(Cit)^{I3}YQ(Pen)Y | 24 | 11-50 |
| 5409 | | | |
| RgIA- | G(Pen)CTDPR(Pen) | >1000 | ND^{b} |
| 5493 | (Cit)^{I3}YQCY | | |
| RgIA- | GC(Pen)TDPRC | >33 | ND |
| 5446 | (Cit)^{I3}YQ(Pen)Y | | |
| RgIA- | GC(Pen)TDPRCR^{I3}YQCY | 0.39 | 0.27-0.59 |
| 5432^{c} | | | |

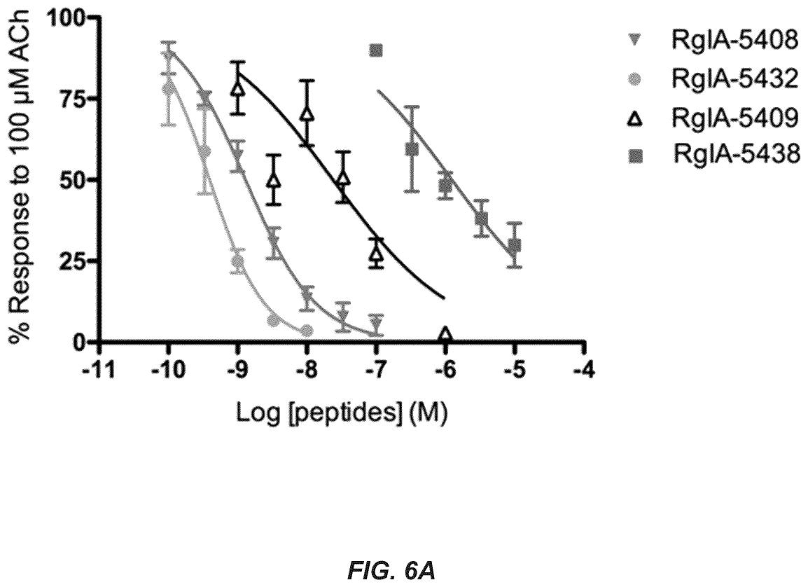

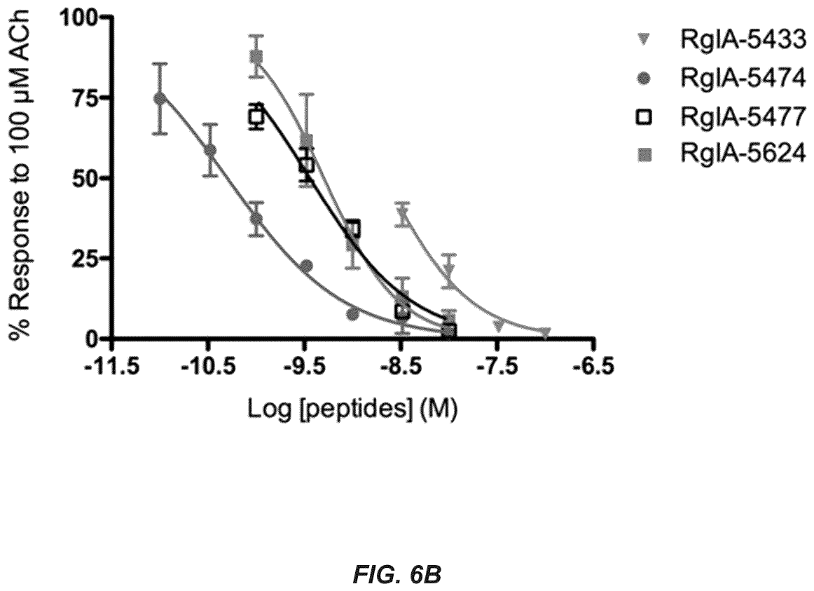

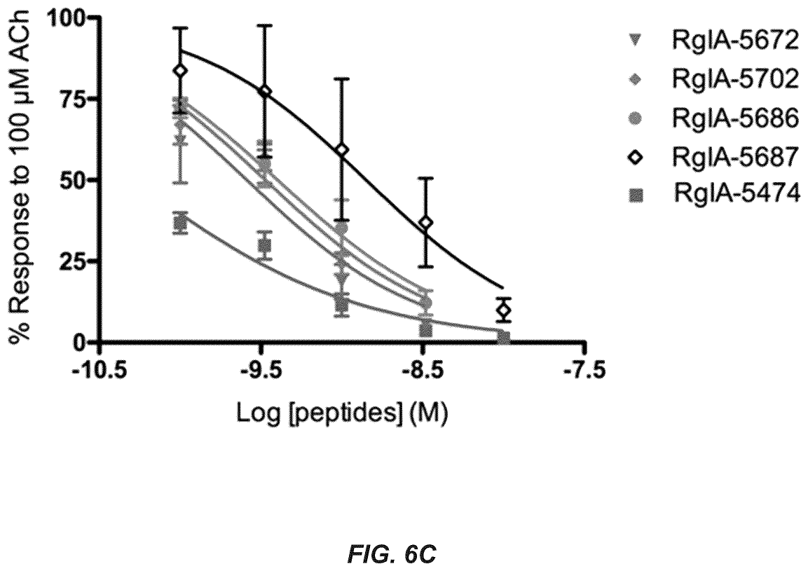

^{a}Data was collected by applying 100 μM ACh to Xenopus oocytes heterologously expressing the receptor. IC₅₀ and 95% confidence intervals (CI) are expressed as the mean ± SEM from more than three separate oocytes. Ba²⁺ containing ND-96 buffer was utilized unless otherwise noted. Underlined residues indicate changes introduced into the sequence in this series of analogs. Amino acid abbreviations: Pen, L-penicillamine; Cit, L-citrulline, ^{I3}Y, 3-iodo-L-tyrosine. The C-terminus of each peptide depicted in this table is a free carboxyl group.
^{b}ND, not determined.
^{c}Data was collected in Ca²⁺ containing ND-96 buffer. Concentration-response curves and Hill slope values are shown in FIG. 6A and Table 4B.

TABLE 4B
Hill slope values for blockade of human (h) and
rat (r) α9α10 and human α7 nAChRs.

| Analog | subtype of nAChR | Hill slope values] |
|---|---|---|
| RgIA-5439 | h α9α10 | 0.5 ± 0.1 |
| RgIA-5408 | h α9α10 | 0.85 ± 0.09 |
| RgIA-5409 | h α9α10 | 0.51 ± 0.2 |
| RgIA-5432 | h α9α10 | 1.09 ± 0.2 |
| RgIA-5624 | h α9α10 | 1.1 ± 0.2 |
| RgIA-5433 | h α9α10 | 0.97 ± 0.2 |
| RgIA-5477 | h α9α10 | 0.83 ± 0.08 |
| RgIA-5474 | h α9α10 | 0.73 ± 0.08 |
| RgIA-5474 | r α9α10 | 2.7 ± 0.8 |
| RgIA-5474 | h α7 | 1.3 ± 0.1 |
| RgIA-5702 | h α9α10 | 0.81 ± 0.1 |
| RgIA-5672 | h α9α10 | 0.82 ± 0.2 |
| RgIA-5686 | h α9α10 | 0.78 ± 0.1 |
| RgIA-5687 | h α9α10 | 0.82 ± 0.3 |
| RgIA-5711 | h α9α10 | 0.83 ± 0.2 |

The position of the Pen residue affected peptide potency. Substituting 2Cys with L-Pen in the 2Cys-8Cys disulfide bridge provided an 800× reduction in potency on the human α9α10 nAChR compared to RgIA4 (RgIA5439 IC₅₀>1200 nM vs. RgIA4 IC₅₀=1.5 nM). Similarly, substituting 8Cys with L-Pen in the 2Cys-8Cys disulfide bridge provided a 600× reduction in potency on the human α9α10 nAChR compared to RgIA4 (RgIA5440 IC₅₀>1000 nM vs. IC₅₀=1.5 nM). A comparable effect was observed when both substituting 2Cys with L-Pen in the 2Cys-8Cys disulfide bridge and substituting 8Cys with L-Pen in the 2Cys-8Cys disulfide bridge (RgIA-5493 IC₅₀>1000 nM). The disulfide bridge between 2Cys and 8Cys has been speculated to interact between α-conotoxins and neuronal nA-ChRs. The bulky β-methyls of Pen might disrupt that interaction. NMR studies can determine what conformational effects are induced by the two methyl groups of the Pen residue.

In contrast, substituting Cys with Pen in the 3Cys-12Cys disulfide bridge provided lower reductions in potency. Substituting 12Cys in the 3Cys-12Cys disulfide bridge yielded an approximately 16-fold loss of potency (RgIA-5409 IC₅₀=24 nM vs. RgIA4 IC₅₀=1.5 nM). A comparable reduction in potency was observed when substituting both 3Cys and 12Cys in the 3Cys-12Cys disulfide bridge (RgIA-5446 IC₅₀>33 nM).

However, substitution with L-Pen in position 3 led to the RgIA-5408 analog that retained low-nanomolar affinity (RgIA-5408 IC_{50=1.3} nM). This trend was also observed when 3Cys was substituted with L-Pen in RgIA5.22, which is another analog of RIA. The resulting peptide, RgIA-5432, blocked the human α9α10 nAChRs equipotent to RgIA5 (RgIA-5432 IC₅₀=0.39 nM and RgIA5 IC₅₀=0.44 nM).

Substitution of Cys with Pen provided varying effects based on which specific Cys residue was substituted. Substituting Cys with Pen at three of the four positions caused large losses in desired activity. In contrast, substitution of 3Cys with Pen preserved the desired activity. This indicates that selective Cys substitution can facilitate a desired high affinity. The observed increase in potency of RgIA-5474 also appeared to be a result of the presence of Pen in the sequence. To confirm this, an analog of RgIA-5474 was synthesized in which the 3Pen residue was replaced by 3Cys.

### Example 5

### Effect of Side Chain Orientation on IC₅₀

The spatial orientation of each cysteine's side chain also plays a role. Individually substituting each L-Cys with D-Cys in the native RgIA led to inactive analogs on human α9α10 nAChRs when 2Cys or 12Cys were mutated (IC₅₀>10 μM). In addition, individually 25 substituting each L-Cys with D-Cys in the native RgIA led to analogs with drastic decreases in potency when 3Cys and 8Cys were mutated (IC₅₀=6440 nM and IC₅₀=6950 nM, respectively). Similar effects were observed when each individual Cys in the sequence was replaced with D-Pen. The resulting analogs showed little or no activity on human α9α10 nAChRs at 10 μM.

### Example 6

### Effect of C-Terminal Substitutions on IC₅₀

Since the RgIA5 Pen-containing analog RgIA-5432 was the most potent of the initially tested group of analogs it was used to design the next set of peptides. The incorporation of Pen residues caused increased hydrophobicity of 3Pen RgIA-5432, and caused it to be prone to precipitation. The addition of a positively charged L-Arg at the C-terminal position 14 increased the peptide's solubility without affecting potency, as shown in Table 6A. When both 13Tyr and 14Arg were substituted by D-Tyr and D-Arg, an approximate 4× drop in potency was observed for RgIA-5433, which indicated that the spatial orientation of the C-terminal residues affects the peptide's ability to bind to the receptor.

TABLE 6A
C-terminal substitutions and their impact
on the potency of RgIA-5474 on blocking
human α9α10 nAChRs.

| | | IC₅₀ª | 95% CI |
|---|---|---|---|
| Analog | Sequence | (nM) | (nM) |
| RgIA- | GC(Pen)TDPRCR^{I3}YQCYR | 0.51 | 0.34-0.75 |
| 5624^{b} | | | |
| RgIA- | GC(Pen)TDPRCR^{I3}YQCyr | 2.1 | 1.4-3.2 |
| 5433 | | | |
| RgIA- | GC(Pen)TDPRCR^{I3}YQC | 0.0504 | 0.037-0.069 |
| 5474 | (β³hY)R | | |
| RgIA- | GC(Pen)TDPRCR^{I3}YQC | 0.36 | 0.28-0.46 |
| 5477 | (β³hY)r | | |

^{a}Data was collected by applying 100 μM ACh to Xenopus oocytes heterologously expressing the receptor. IC₅₀ and 95% confidence intervals (CI) are expressed as the mean ± SEM from 3-5 separate oocytes. Ba²⁺ containing ND-96 buffer was utilized. Underlined residues indicate changes introduced into the sequence in this series of analogs. Amino acid abbreviations: Pen, L-penicillamine; ^{I3}Y, 3-iodo-L-tyrosine; y, D-Tyr; r, D-Arg; β³hY, β³-homo tyrosine. The C-terminus of each peptide depicted in this table is a free carboxyl group.
^{b}Data was collected in Ca²⁺ containing ND-96 buffer. Concentration-response curves and the Hill slope values are shown in FIG. 6B and Table 4B.

A RgIA structure-activity relationship (SAR) study indicated that residues in the first loop: 5Asp, 6Pro, and 7Arg were responsible for orienting the peptide for binding to the nAChR and 9Arg and for facilitating selectivity towards the α9α10 subtype of nAChR. SAR data also revealed that an Arg13Tyr mutation can enhance RgIA potency. B-amino acids are homologs of natural α-amino acids, with an extra methylene group immediately before the carboxylic group in the backbone of the amino acids. The side chain can be positioned either on the α- or β-carbon (β2- or β3-analog, respectively) or can be present in both positions. In order to maintain the side-chain position of 13Tyr, this residue was substituted with β3-homo tyrosine (β3hY) to synthesize the analog RgIA-5474 with an IC₅₀=0.0504 nM. The changes present in the RgIA-5474 analog facilitate a 9000× increased potency over the native RgIA on the human α9α10 nAChR.

At saturating concentrations (10 nM), RgIA-5474 was characterized by slow off-rate kinetics compared to RgIA, with 3% (+1.7) recovery after 5 min peptide washout compared to >99% recovery for RgIA at 10 μM (data not shown). These results indicate that the 14Arg residue confers high affinity, in part, via modulation. When D-Arg was incorporated in position 14 the potency dropped 7× (IC₅₀=0.36 nM), as shown in Table 6A, compared to the all-L analog, which indicated that the positioning of the 14Arg side chain within the receptor can affect potency.

### Example 7

### Effect of C-Terminal Charge on IC₅₀

In order to investigate the role of C-terminal Arg, a series of RgIA-5474 analogs were prepared in which 14Arg was substituted by other positively charged, neutral, or negatively charged amino acids, as shown in Table 7A. Substitution with 14Lys and 140m (omithine, a non-proteinogenic amino acid with a side chain one methylene group shorter than Lys that terminates with a primary amine) resulted in analogs with a 7- and 5-fold drop in potency, respectively. A neutral 14Cit (citrulline, an uncharged arginine analog, terminating in a carbamoyl amino group) substitution led to an 8× potency reduction. Thus, the addition of 14Arg may enable formation of one or more hydrogen-bonds via its guanidinium group with receptor residues at or adjacent to the core binding site. Substitution with a negatively charged Glu in position 14 caused an approximate 28× reduction in potency with an IC₅₀ of 1.4 nM for RgIA-5687.

TABLE 7A
The role of charge in position 14 of RgIA-
5474 on blocking human α9α10 nAChRs.

| | | IC₅₀ | 95% CI |
|---|---|---|---|
| Analog | Sequence | (nM)^{a} | (nM) |
| RgIA- | GC(Pen)TDPRCR^{I3}YQC | 0.051 | 0.027-0.095 |
| 5474 | (ß³hY)R | | |
| RgIA- | GC(Pen)TDPRCR^{I3}YQC | 0.34 | 0.24-0.47 |
| 5702 | (ß³hY)K | | |
| RgIA- | GC(Pen)TDPRCR^{I3}YQC | 0.26 | 0.15-0.45 |
| 5672 | (ß³hY)Orn | | |
| RgIA- | GC(Pen)TDPRCR^{I3}YQC | 0.40 | 0.27-0.601 |
| 5686 | (ß³hY)Cit | | |
| RgIA- | GC(Pen)TDPRCR^{I3}YQC | 1.4 | 0.57-3.5 |
| 5687 | (ß³hY)E | | |

^{a}Data was collected by applying 100 μM ACh to Xenopus oocytes heterologously expressing the receptor. IC₅₀ and 95% confidence intervals (CI) are expressed as the mean ± SEM from 3-5 separate oocytes. Ca²⁺ containing ND-96 buffer was utilized. Underlined residues indicate changes introduced into the sequence in this series of analogs. Amino acid abbreviations: Pen, L-penicillamine; ^{I3}Y, 3-iodo-L-tyrosine; β³hY, β³-homo tyrosine; Orn, L-ornithine; Cit, L-citrulline. The C-terminus of each peptide depicted in this table is a free carboxyl group. Concentration-response curves and the Hill slope values are shown in FIG. 6C and Table 4B.

The combined data indicated that there are several components contributing to the high potency of RgIA-5474: (i) the mutations introduced into the first (Ser4Thr) and second (Tyr1013Tyr, Arg11Gln) loop of the peptide, (ii) the C-terminal modification (Arg13Tyr), and (iii) in the set of analogs, β3-homo tyrosine positioned the side chain of 14Arg by one methylene group farther from the peptide's core which facilitated increased interaction between the guanidinium group and the channel.

### Example 8

### Stability of RoIA4 and RIA-5474 in Human Serum

### Methods—Serum-Stability Assay.

150 nmols of RgIA4 and RgIA-5474 were dissolved in 250 μL water and this solution was added into 750 μL of human serum solution (from human male AB plasma, sterile filtered and defrosted once and pre-centrifuged 15 min to remove lipids, 250 μL of supernatant was added to 500 μL water: Millipore Sigma) to produce a total solution of 25% human serum. Twenty-five percent serum concentration was utilized to confirm that the reaction speed was linearly dependent on the serum concentration and that the peptide concentration was not rate limiting. The final concentration of the peptides in the solution was 150 μM. The peptide-serum solution was incubated at 37 C and an individual 100 μL sample of the solution was removed at 24 h, treated with 50 μL ice cold acetonitrile (HPLC grade), and cooled on ice for 15 mins. The suspension was then centrifuged at 13,000 rpm for 5 mins at room temperature. Next 10 μL of the supernatant was removed and diluted in 90 μL of buffer A (0.1% TFA in water) to produce the HPLC sample.

The samples were analyzed by RP-HPLC with a linear gradient of 10-50% buffer B over 40 mins. Peptide peak areas were integrated at 280 nm and the percent of peptide left 20) was compared to the initial peptide was graphed. The serum stability experiments were repeated 2 times in triplicates for each peptide. The synthesis of the reference peptide RgIA4 was described previously and the ribbon RgIA4 was synthesized following the protocol described for RgIA4, and was obtained with 99% purity (RT and MS data are provided in Table 3A). The ribbon RgIA-5474 was prepared synthetically, following the protocol described for RgIA-5474, with the disulfide bridge formed in the following order: 3Pen-8Cys first and 2Cys-12Cys second. The ribbon RgIA-5474 was obtained with 99% purity (RT and MS data are provided in Table 3A).

### Results:

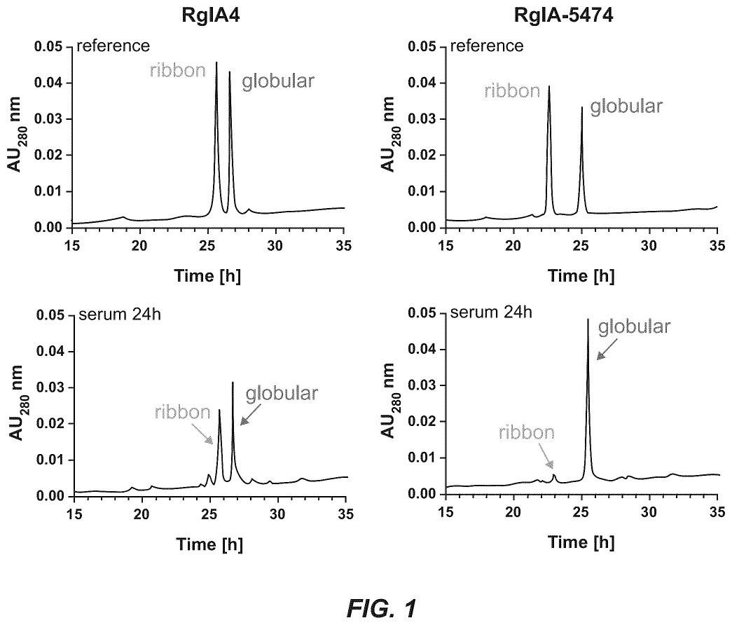

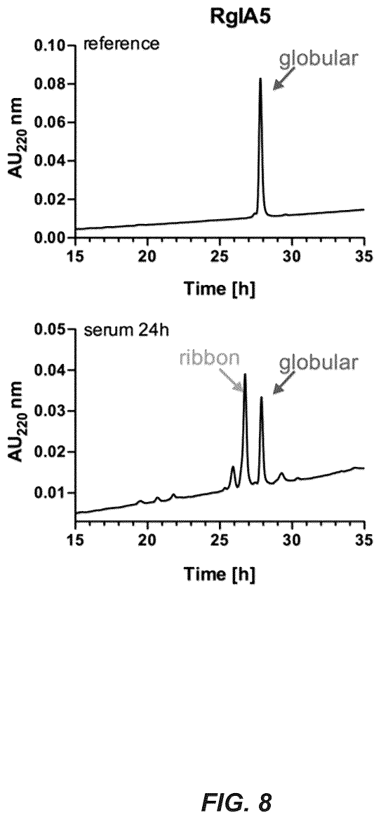

30) The stability of RgIA-5474 in human serum was compared to RgIA4. Twenty-five percent serum concentration was used to provide a reaction speed that was linearly dependent on the serum concentration and to confirm that the peptide concentration was not rate limiting. FIG. 1 illustrates the stability of RgIA4 and RgIA-5474 in 25% human serum. In the top panel, HPLC traces of a mixture (˜1:1) of the globular and ribbon forms of the reference peptides RgIA4 and RgIA-5474 is shown. In the bottom panel. The serum stability at 24h (n=6) is shown. The reaction was monitored by RP-HPLC using a C18 column and a gradient ranging from 10% to 50% of buffer B in 40 min with a flow rate of 1 mL/min. Serum stability data for RgIA5 is shown in FIG. 8.

As illustrated in FIG. 1, RgIA-5474 retained its structural integrity with 94% (=0.7) in the globular form and 6% (+0.7) in the ribbon form present at 24 h, whereas substantial disulfide reshuffling of RgIA4 occurred with 64% (+0.5) in the globular form and 36% (+0.5) inthe ribbon form present (as calculated based on the area under the HPLC peak, n=6). This data indicated a protective effect of the penicillamine on the disulfide bridge in which the β,β-dimethyl groups provided steric hindrance and slowed down the reshuffling process.

### Example 9

### Selectivity of RoIA-5474 for α9α10 nAChR

### Methods: Receptor Pharmacology

Synthetic RgIA-5474 was tested on non-nAChRs targets in the CYP Inhibition assays by Eu-rofins Cerep, Pharma Discovery Service, Celle l'Evescault, France. Automated patch-clamp electrophysiology was used for assessing the human ether-a-go-go-related gene (hERG) function. RgIA-5474 was also tested at the Dept. of Veterans Affairs Medical Center Research Service (R&D-22) in Portland, OR on serotonin, dopamine, and opioid receptors, as well as biogenic amine transporters. Detailed description of the assays and the data are presented in the Tables 9B-9F.

### Selectivity:

Analog RgIA-5474 was the most active peptide from the series of analogs synthesized and was highly selective for the α9α10 nAChR. The RgIA-5474 peptide was tested on several different subtypes of the human and rat nAChR, as shown in Table 9A, and was found to be inactive (IC₅₀>10,000 nM) on all subtypes tested with the exception of the human 7 nAChR, for which it had 2000× lower potency compared to the human α9α10 nAChR. RgIA-5474 retained its potency for rat α9α10 nAChR with an IC₅₀ of 0.39 nM, and a significant decrease in potency was observed for rat 7. RgIA-5474 was additionally tested against μ-, δ- and κ-opioid receptor subtypes, N- and L-type Ca2+ channels, serotonin-, and norepinephrine-transporters, and a wide panel of relevant receptors and ion channels and showed low or no activity at micromolar levels, as depicted in Tables 9B to 9F. Thus, RgIA-5474 is a highly selective peptide.

TABLE 9A
Subtype selectivity of RgIA-5474.

| | | |
|---|---|---|
| Subtypes | Human IC₅₀ [nM] | Rat IC₅₀ [nM] |
| of nAChR | (95% CI)^{a} | (95% CI)^{a} |

| | | | |
|---|---|---|---|
| α9α10 | 0.051 | (0.027-0.095) | 0.39 (0.33-0.48) |

| | | |
|---|---|---|
| α2β2 | >10,000 | >10,000 |
| α2β4 | >10,000 | >10,000 |
| α3β2 | >10,000 | >10,000 |
| α3β4 | >10,000 | >10,000 |
| α4β2 | >10,000 | >10,000 |
| α4β4 | >10,000 | >10,000 |
| α6/α3β4 | >10,000 | >10,000 |
| α6/α3β2β3 | >10,000 | >10,000 |

| | | | |
|---|---|---|---|
| α7 | 115 | (102-130) | >333 |

| | | |
|---|---|---|
| α1β1δε | >10,000 | NA^{b} |

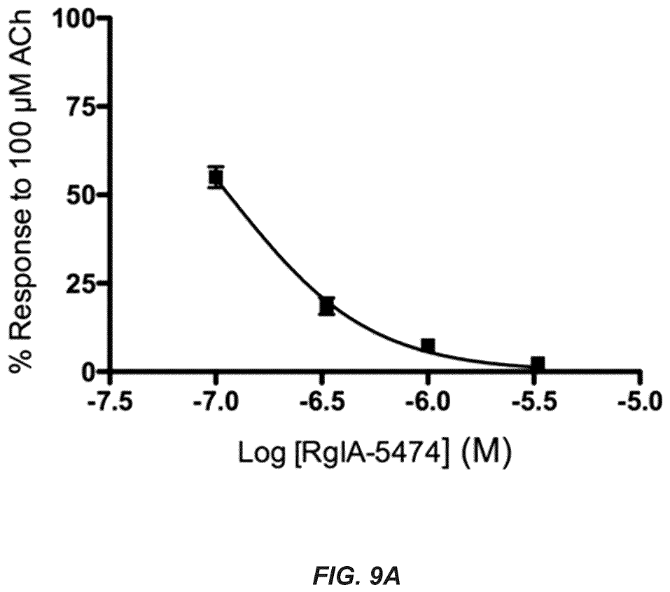

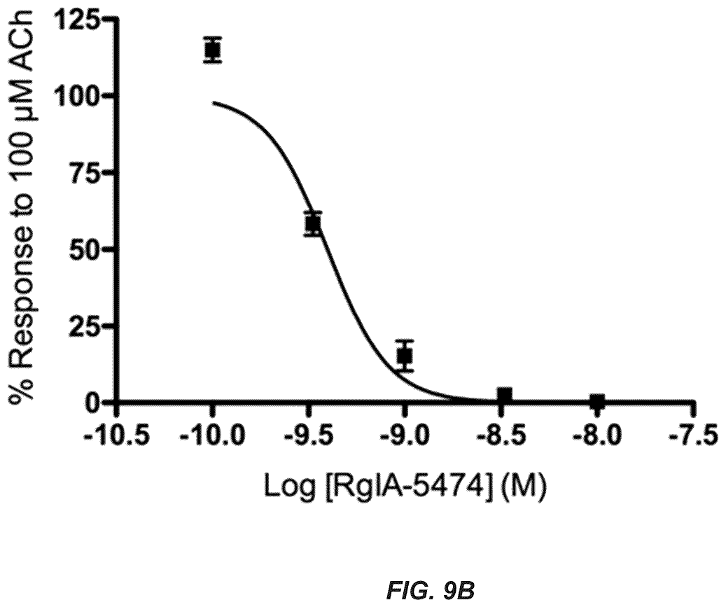

^{a}Data was collected in Ca²⁺ containing ND-96 buffer and 100 μM ACh application. IC₅₀ and 95% confidence intervals (CI) are expressed as the mean ± SEM from 3-5 separate oocytes;
^{b}peptide was not tested on this subtype. Concentration-response curves and the Hill slope values are shown in FIGS. 9A and 9B and Table 4B.

Binding Activity of rgia-5474 on Ion Channels, Receptors and Transporters

Synthetic RgIA-5474 was tested on non-nAChRs in binding assays by Eurofins Cerep, Pharma Discovery Service, Celle l′Evescault, France. The peptide was screened at 0.1 μM and 10 μM and each experiment was conducted in duplicate. Shown in Table 9B are results for 10 μM (average of 2 runs/ mean), except for nicotinic neuronal a7, for which a result at 100 nM also was reported (*). Kd concentrations of indicated radioligands were used. Stably or transiently transfected recombinant cell lines expressing human channel receptors or transporters were used unless otherwise indicated (rat cerebral cortex, cerebellum or spinal cord were used). The binding was calculated as a percentage inhibition of the binding of a radioactively labeled ligand specific for each target. An inhibition or stimulation higher than 50% was considered to represent significant effects. Automated patch-clamp electrophysiology was used for assessing the human ether-a-go-go-related gene (hERG) function to predict potential cardiac toxicity and the peptide was tested at 10 nmol, 100 nmol, 1 μM, 10 μM and 100 μM in duplicate for IC₅₀ determination, as shown in Table 9C. N.C. indicates not calculated. Fluorimetry was used to assess agonistic and antagonistic activity of RgIA-5474 on human GABAB1b receptors, employing human recombinant RBL cells.

Two separate experiments were performed and the peptide was applied at 8 different concentrations ranging from 3 nM to 10 μM, for the functional IC₅₀ and EC₅₀ determination, as shown in Table 9D. The effect was calculated as a percentage of control response to a known reference agonist or antagonist. An inhibition or stimulation higher than 50% was considered to represent significant effects. N.C. indicates not calculated.

RgIA-5474 was also tested in a cytochrome P450 Inhibition assay at 0.1 μM and 10 μM. Table 9E displays results for 10 μM and an average value for 2 runs.

Finally, RgIA-5474 was tested at the Dept. of Veterans Affairs Medical Center Research Service (R&D-22) in Portland, OR on serotonin, dopamine, and opioid receptors, as well as biogenic amine transporters, as shown in in Table 9F. For each receptor, the peptide was tested in two independent experiments, with each conducted with at least triplicate determinations. Data is expressed as the percentage inhibition of specific control binding. Numbers represent, except where noted, means±range from two independent experiments, each conducted with at least triplicate determinations.

TABLE 9B
ACTIVITY OF RGIA-5474 ON NON-NACHRS IN A BINDING ASSAY.

| Target | Radioligand | Inhibition [%] |
|---|---|---|
| Adenosine A₁ | [³H]DPCPX | 0.2 |
| Adenosine A_{2A} | [³H]CGS 21680 | −15.7 |
| Adenosine A_{2B} | [³H]CPX | 4 |
| Adenosine A₃ | [¹²⁵I]AB-MECA | 17 |
| α₁ adrenergic (non-selective) (rat) | [³H]prazosin | 8.7 |
| α₂ adrenergic (non-selective) (rat) | [³H]RX 821002 | 0 |
| β₁ adrenergic | [³H](−)CGP12177 | 19.5 |
| Angiotensin AT₁ | [¹²⁵I][ Sar¹, Ile⁸]-AT-II | −13.6 |
| Angiotensin AT₂ | [¹²⁵I]CGP 42112A | 17.8 |
| Benzodiazepine (central) (rat) | [³H]flunitrazepam | −6.4 |
| β2 adrenergic | [³H]bradykinin | 4.8 |
| Cannabinoid CB1 | [³H]CP55940 | 27.6 |
| Cannabinoid CB2 | [³H]WIN 55212-2 | −8.3 |
| Cholecystokinin CCK₁ | [¹²⁵I]CCK-8s | −25.1 |
| Cholecystokinin CCK₂ | [¹²⁵I]CCK-8s | 11.8 |
| Corticotropin-releasing factor CRF₁ | [¹²⁵I]sauvagine | −5.3 |
| Dopamine D_{4.4} | [³H] methyl-spiperone | −4.8 |
| Endothelin ET_{A} | [¹²⁵I]Endothelin-1 | −17.9 |
| Endothelin ET_{B} | [¹²⁵I]Endothelin-3 | −5.3 |
| γ-aminobutyric acid GABA_{A1} (h) (α1, β2, γ2) | [³H]muscimol | 0.8 |
| γ-aminobutyric acid GABA_{B(1b)} | [³H]CGP 54626 | −6.4 |
| Galanin GAL₁ (h) | [¹²⁵I]galanin | −14.2 |
| Galanin GAL₂ | [¹²⁵I]galanin | −4.7 |
| Glutamate AMPA site (rat) | [³H]AMPA | −4.5 |
| Glutamate kainite site (rat) | [³H] kainic acid | −19.9 |
| Glutamate NMDA site (rat) | [³H] CGP 39653 | −0.1 |
| Metabotropic Glutamate receptor 1 (rat) | [³H] Quisqualate | −4.0 |
| Metabotropic Glutamate receptor 5 | [³H] Quisqualate | 7.7 |
| glycine (strychnine-sensitive) | [³H] strychnine | 22.4 |
| glycine (strychnine-insensitive) (rat) | [³H]MDL 105,519 | 14.9 |
| Histamine H₁ | [³H] pyrilamine | −0.2 |
| Histamine H₂ | [¹²⁵I]APT | −6.7 |
| Histamine H₃ | [³H] N^{α}-Me-histamine | 24.1 |
| Leukotriene CysLT₁ (LTD₄) | [³H LTD₄ | −3.2 |
| Muscarinic (non-selective) (rat) | [³H]QNB | −0.4 |
| Muscarinic M1 | [³H] pirenzepine | 7.6 |
| Muscarinic M2 | [³H]AF-DX 384 | 27.2 |
| Muscarinic M3 | [³H]4-DAMP | −2.0 |
| Neurokinin NK₁ | [¹²⁵I]substance P | −7.5 |
| Neurokinin NK₂ | [¹²⁵I]NKA | 41.6 |
| Neurokinin NK₃ | [¹²⁵I]SR 142801 | −35.5 |
| Nicotinic neuronal α4β2 | [³H]cytisine | −18.4 |
| Nicotinic neuronal α7 | [¹²⁵I]α-bungarotoxin | 54.3 (−3.1)* |
| Nicotinic muscle-type | [¹²⁵I]α-bungarotoxin | −42.3 |
| Opioid δ2 (DOP) | [³H]DADLE | 4.5 |
| Opioid κ (KOP) (rat) | [³H]U 69593 | 2.6 |
| Opioid μ (MOP) | [³H]DAMGO | 14.6 |
| Oxytocin | [³H]oxytocin | 13.1 |
| Opioid NOP (ORL1) | [³H]nociceptin | 2.7 |
| Platelet activating factor | [³H]C₁₆-PAF | 9.1 |
| Glutamate Phencyclidine PCP site (Rat) | [³H]TCP | −6.0 |
| Glucocorticoids receptor | [³H]dexamethasone | 0.6 |
| Estrogen receptor (non-selective) | [³H]estradiol | −14.8 |
| Androgen Receptor | [³H]methyltrienolone | 3.4 |
| Thyrotropin-releasing hormone TRH₁ | [³H]Me-TRH | −8.7 |
| Vasoactive intestinal peptide 2 (VPAC₂) | [¹²⁵I]VIP | 0.7 |
| Vasoactive intestinal peptide 1 (VPAC₁) | [¹²⁵I]VIP | 3.4 |
| Vasopressin V₁ₐ | [¹²⁵I]AVP | 4.2 |
| Vasopressin V_{1b} | [¹²⁵I]AVP | −6.4 |
| Ca²⁺ channel (L type, dihydropyridine site)(rat) | [³H]nitrendipine | −10.1 |
| Ca²⁺ channel (L type, ditiazem site) | [³H]diltiazem | −16.5 |
| (benzothiazepines) (rat) | | |
| Ca²⁺ channel (N type) (rat) | [¹²⁵I]ω-conotoxin GVIA | −7.3 |
| ATP sensitive potassium channel (rat) | [³H]glibenclamide | −25.8 |
| Potassium channel SK_{Ca} (rat) | [¹²⁵I]apamin | 14.6 |
| Sodium Channel (site 2) (rat) | [³H]batrachotoxin | 7.2 |

TABLE 9C
ACTIVITY OF RGIA-5474 IN THE HERG CHANNEL ASSAY.

| | IC₅₀ | IC₅₀ mean | IC₅₀ range |
|---|---|---|---|
| Assay | (nM) | (nM) | (nM) |
| hERG IC50 (hERG-CHO, | >100,000 | N.C. | N.C. |
| automated patch-clamp) | | | |

TABLE 9D
ACTIVITY OF RGIA-5474 ON
HUMAN GABAB1B RECEPTOR.

| | antagonist effect | agonist effect |
|---|---|---|
| Assay | IC₅₀ (nM) | EC₅₀ (nM) |
| GABAB1b | N.C. | N.C. |
| (Fluorimetry) | | |

TABLE 9E
ACTIVITY OF RGIA-5474 IN THE CYTOCHROME
P450 INHIBITION ASSAY.

| Target: | | |
|---|---|---|
| Cytochrome | | Inhibition |
| P450 | Substrate (recombinant) | [%] |
| CYP1A2 | 3-cyano-7ethoxycoumarin (CEC) | −3.8 |
| CYP2A6 | Coumarin | 0.3 |
| CYP2C9 | 7-methoxy-4-trifluoromethylcoumarin (MFC) | 3.1 |
| CYP2C19 | 3-cyano-7ethoxycoumarin (CEC) | −1.9 |
| CYP2D6 | 7-methoxy-4-trifluoromethylcoumarin (MFC) | −1.6 |
| CYP3A4 | 7-benzyloxy-4-trifluoromethylcoumarin (BFC) | −2.0 |

TABLE 9F
ACTIVITY OF RGIA-5474 ON SEROTONIN,
DOPAMINE AND OPIOID RECEPTORS,
AND BIOGENIC AMINE TRANSPORTERS.

| Target | Radioligand | Inhibition [%] |
|---|---|---|
| Serotonin receptor 5-HT1A | [³H]8OH-DPAT binding | 3.5 ± 9.9 |
| Serotonin receptor 5-HT2A | [³H]5-HT binding | 11 ± 17 |
| Serotonin receptor 5-HT2C | [³H]5-HT binding | 12.5 ± 2.2 |
| Opioid δ2 receptor (DOR) | [³H]DPDPE | 3.47 ± 0.46 |
| Opioid κ receptor (KOR) | [³H]U69593 | −7.33 ± 0.54 |
| Opioid μ receptor (MOR) | [³H]DAMGO | −2.38 ± 0.47 |
| Dopaminergic D1 | [³H]SCH23390 | −0.10 ± 5.28 |
| Dopaminergic D2 | [³H]YM-09151-2 | 1.5 ± 4.0 |
| Dopaminergic D3 | [³H]YM-09151-2 | 0.48 ± 6.76 |
| Dopaminergic D4.4 | [³H]YM-09151-2 | −0.86 ± 3.44 |
| Dopamine transporter | [¹²⁵I]RTI-55 | 7.0 ± 7.2 |
| Serotonin receptor 5-HT2ce | [¹²⁵I]RTI-55 | 7.1 ± 1.2 |
| Norepinephrine transporter | [¹²⁵I]RTI-55 | 6.1 ± 6.2 |

### Example 10

### Analgesic Activity of RIA-5474

### Methods—Oxaliplatin-Induced Peripheral Neuropathy in Mice.

Neuropathy in mice was induced. Oxaliplatin (MedChem Express, Monmouth Junction, NJ) was dissolved at 0.875 μg/μL in 0.9% NaCl, sterile filtered. RgIA-5474 was dissolved at 0.01 and 0.001 μg/μL in 0.9% NaCl, sterile filtered. Mice were divided into four equally sized groups (n=8 animals) and injected with saline (i.p.)+saline (s.c.), oxaliplatin (3.5 mg/kg i.p.)+saline (s.c.), oxaliplatin (3.5 mg/kg. i.p.)+RgIA-5474 (40 μg/kg. s.c.), and oxaliplatin (3.5 mg/kg. i.p.)+RgIA-5474 (4 μg/kg, s.c.). Mice were injected once per day on Wednesday, Thursday, and Friday, and again on Monday and Tuesday. Mice were injected 5 times per week rather than 7 times per week, for convenience, based on prior data with a related compounds. It was not assessed if the analgesic effect was enhanced with a 7 times vs. 5 times per week dosing. Mice were tested on Wednesdays before daily injection occurred (24-h post last injection). This injection pattern was repeated for an additional 2 weeks until the 22nd day: and then mice were tested weekly on Wednesdays to track the effects. Testing was conducted using a cold-plate test chamber (IITC, Inc. Life Science). Animals were allowed to acclimate in the chamber at room temperature (23° C.) for 2-5 min. Temperature was then lowered at a rate of 10° C.per minute. The testing was stopped when the animal lifted both forepaws and shaking or licking occurred. Alternating lifting of forepaws was not scored. Throughout the study period, experimenters were blinded as to the identity of the injected compounds. Data was analyzed with one-way ANOVA using Dunnett's multiple comparison test (GraphPad Prism, GraphPad Software, San Diego, CA).

### Results:

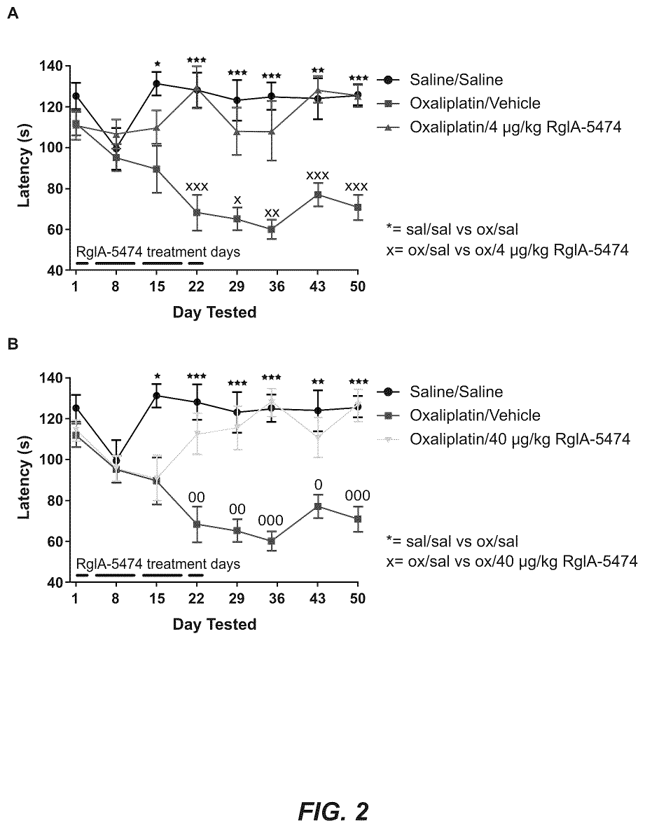

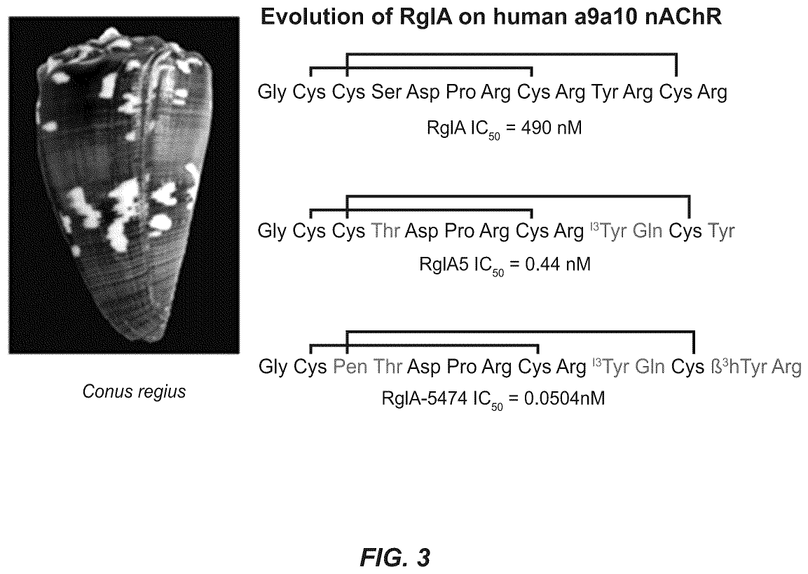

RgIA-5474 was analyzed for analgesic activity, as shown in FIG. 2. Mice were injected i.p. with oxaliplatin (Ox: 3.5 mg/kg) daily as described herein. Control animals were treated with vehicle. RgIA-5474 was dissolved in sterile saline (sal) and injected s.c. daily. Withdrawal latency was used as a measure of cold allodynia. The cold allodynia test was performed on days 8, 15, and 22 at 24 h after RgIA-5474 administration at 4 μg/kg (panel A) and 40 μg/kg (panel B). Values are expressed as the mean±SEM from eight mice for each experimental determination. ***P<0.001, **P<0.01, *P<0.05 significantly different from vehicle.

Chemotherapy-induced peripheral neuropathy is a side effect of platinum-containing drugs used to treat various cancers. The consequent pain can be disabling and is associated with a significant increase in opioid use. In many cases, the pain is partially reversible, but the symptoms may last for years and the neuronal damage can be permanent. At present, there are no approved drugs to prevent or treat neuropathy, often forcing premature cessation of chemotherapy treatment. RgIA-5474 was tested using a mouse oxaliplatin-induced peripheral neuropathy model. RgIA-5474 successfully reversed chemotherapy-induced cold-allodynia or painful cold sensitization.

The opioid class of medications can lead to the development of tolerance. Chronic use of opioids results in decreased therapeutic effect and the use for a higher dose to produce the initial analgesia. This phenomenon frequently leads to accidental overdose when an opioid is stopped and is subsequently re-initiated at the higher dose. Tolerance was not observed with a constant dose of RgIA-5474. The therapeutic effect was not apparent until after 15 doses of drug were given over a period of three weeks. Furthermore, the therapeutic effect of RgIA-5474 persisted for four-weeks after cessation of treatment.

Selective incorporation of penicillamine and B3-homo tyrosine increased the activity of the naturally occurring peptide to make it more potent, selective, and resistant to disulfide reshuffling. Therefore, the role of the disulfides on the activity of this class of peptides was validated and indicated additional residues contributing to the potency on the α9α10 nAChRs. Thus, RgIA-5474 is a peptide with a high potency, selectivity, and a non-opioid mechanism of action.

### Example 11

### Voltage Clamp Recording

### Methods—Two-Electrode Voltage-Clamp Recording.

Xenopus laevis (Xenopus 1, Dexter, MI) oocytes were used to heterologously express cloned rat or human nAChR subtypes. Recordings were made 1-5 d post-injection. Oocytes were voltage-clamped at -70 mV at room temperature and pulsed for 1 second, every 60 seconds, with a bolus of acetylcholine, RgIA analogs were flowed on as described herein. Where noted, Ba2+ was substituted for Ca2+ to reduce current run-up following antagonist block. Concentration response curves for the inhibition of α9α10 and a7 nAChR with the Hill slope values are shown in FIGS. 6A to 6C, 7, 9A, and 9B and Table 4B, respectively.

FIG. 6A-6C illustrate concentration-response curves for the inhibition of human α9α10 nAChR. Human α9α10 nAChRs were heterologously expressed in X. laevis oocytes. The potencies of the peptides were assessed using two-electrode voltage clamp electrophysiology as described herein. Data points are the mean±SEM obtained from 3-5 oocytes.

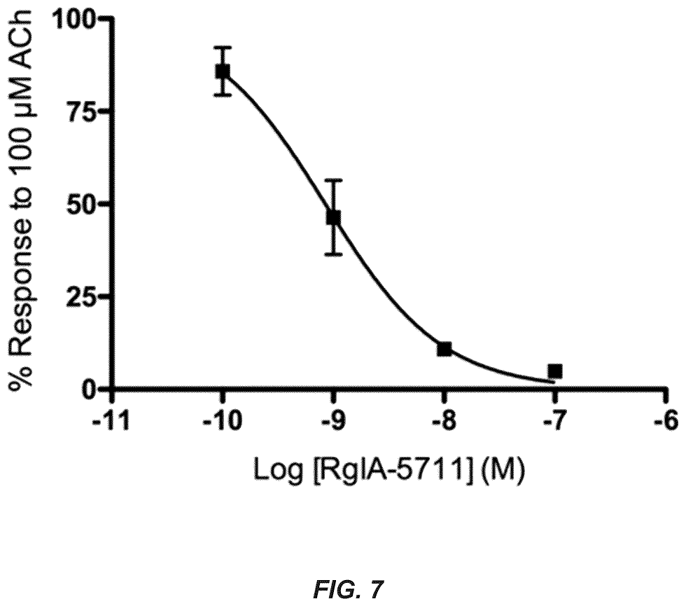

FIG. 7 illustrates a concentration-response curve for the inhibition of human α9α10 nAChRs by RgIA-5711. Rat α9α10 nAChRs were heterologously expressed in X. laevisoocytes and the potency of the peptide was assessed using two-electrode voltage clamp electrophysiology as described herein. Data points are the mean±SEM obtained from 3-5 oocytes.

FIG. 9A illustrates a concentration-response curve for the inhibition of human a7 nAChR by RgIA-5474. Human a7 nAChRs were heterologously expressed in X. laevisoocytes and the potency of the peptide was assessed using two-electrode voltage clamp electrophysiology as described herein. Data points are the mean±SEM obtained from 3-5 oocytes.

FIG. 9B illustrates a concentration-response curve for the inhibition of rat α9α10 nAChRs by RgIA-5474. Rat α9α10 nAChRs were heterologously expressed in X. laevisoocytes and the potency of the peptide was assessed using two-electrode voltage clamp electrophysiology as described herein. Data points are the mean±SEM obtained from 3-5 oocytes.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of blocks might be added to the logical flow for purposes of enhanced utility, accounting, performance, measurement, troubleshooting, or for similar reasons.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

The foregoing detailed description describes the disclosure with reference to specific exemplary embodiments. However, it will be appreciated that various modifications and changes can be made without departing from the scope of the present disclosure as set forth in the appended claims. The detailed description and accompanying drawings are to be regarded as merely illustrative, rather than as restrictive, and all such modifications or changes, if any, are intended to fall within the scope of the present disclosure as described and set forth herein.

1. A synthetic analgesic peptide comprising the amino acid sequence G C X1 T D PRC X2 (R-3-Y) QC X3 X4 (SEQ ID NO: 3), wherein:
X1 is selected from the group consisting of L-penicillamine (L-Pen), D-penicillamine (D-Pen), and L-cysteine,
X2 is selected from the group consisting of L-arginine, D-arginine, or citrulline,
X3 is any amino acid,
X4 is any amino acid, and
R-3-Y is 3-R-tyrosine, wherein 3-R-tyrosine can be a peptide residue selected from the group consisting of 3-chloro-tyrosine, 3-fluoro-tyrosine, 3-iodo-tyrosine, 3-bromo-tyrosine, and tyrosine.

2. The synthetic analgesic peptide of claim 1, wherein X1 is L-Pen.

3. The synthetic analgesic peptide of claim 1, wherein X2 is L-arginine.

4. The synthetic analgesic peptide of claim 1, wherein X3 is selected from the group consisting of beta-homo-tyrosine, L-tyrosine, and D-tyrosine.

5. The synthetic analgesic peptide of claim 1, wherein X4 is selected from the group consisting of L-arginine and D-arginine.

6. The synthetic analgesic peptide of claim 1, comprising the amino acid sequence GCL-Pen TD P R CR (R-3-Y) QC X3 X4 (SEQ ID NO: 4).

7. The synthetic analgesic peptide of claim 1, comprising the amino acid sequence GCL-Pen TDP R CR (1-3-Y) QC X3 X4 (SEQ ID NO: 5).

8. The synthetic analgesic peptide of claim 1, comprising the amino acid sequence GCL-Pen TDP R CR (1-3-Y) Q C (bhY) X4 (SEQ ID NO: 6).

9. The synthetic analgesic peptide of claim 1, comprising the amino acid sequence GCL-Pen TDP R CR (1-3-Y) Q C (3-R-bhY) X4 (SEQ ID NO: 7).

10. The synthetic analgesic peptide of claim 1, comprising the amino acid sequence GCL-Pen TD P R CR (1-3-Y) Q C (bhY) R (SEQ ID NO: 8).

11. The synthetic analgesic peptide of claim 1, comprising the amino acid sequence GCL-Pen TDP R CR (1-3-Y) Q C (3-R-bhY) R (SEQ ID NO: 9).

12. A method for treating or preventing a condition or disorder associated with α9α10 subtype of the nicotinic acetylcholine receptor (nAChR) in a subject comprising administering to the subject a therapeutically effective amount of a composition comprising the peptide of claim 1.

13. The method of claim 12, wherein the condition or disorder is pain.

14. The method of claim 13, wherein the pain is diabetic neuropathy pain.

15. The method of claim 13, wherein the pain is chemotherapy-induced pain.

16-18. (canceled)

19. An RgIA4 peptide analog comprising:
a recognition finger region configured to bind to an α9α10 nicotinic acetylcholine receptor; and
at least two disulfide bridges comprising: an inter-cysteine disulfide bridge between a first cysteine and a second cysteine, and a penicillamine-cysteine disulfide bridge between a first penicillamine and a third cysteine;
wherein the RgIA4 peptide analog has a binding affinity for the α9α10 nicotinic acetylcholine receptor that is at least 50% of a binding affinity of an RgIA4 peptide.

20. The RgIA4 peptide analog of claim 19, further comprising a β³-homo tyrosine bound to the third cysteine.

21. The RgIA4 peptide analog of claim 19, wherein the binding affinity for the α9α10 nicotinic acetylcholine receptor is:
substantially equal to the binding affinity of the RgIA4 peptide, or
greater than the binding affinity of an RgIA4 peptide.

22. The RgIA4 peptide analog of claim 19, wherein the penicillamine-cysteine disulfide bridge provides an increase in potency compared to a potency of an RgIA4 peptide.

23. The RgIA4 peptide analog of claim 19, wherein the RgIA4 peptide analog provides an α9α10 nicotinic acetylcholine receptor IC₅₀ value that is:
at least 25.0× less than the α9α10 nicotinic acetylcholine receptor IC₅₀ value of the RgIA4 peptide, or
at least 10.0× less than the α9α10 nicotinic acetylcholine receptor IC₅₀ value of the RgIA4 peptide, or
at least 5.0× less than the α9α10 nicotinic acetylcholine receptor IC₅₀ value of the RgIA4 peptide, or
at least 2.0× less than the α9α10 nicotinic acetylcholine receptor IC₅₀ value of the RgIA4peptide, or
substantially equal to an α9α10 nicotinic acetylcholine receptor IC₅₀ value of the RgIA4 peptide, or
no greater than 2.0× the α9α10 nicotinic acetylcholine receptor IC₅₀ value of the RgIA4 peptide, or
no greater than 3.0× the α9α10 nicotinic acetylcholine receptor IC₅₀ value of the RgIA4 peptide, or
no greater than 5.0× the α9α10 nicotinic acetylcholine receptor IC₅₀ value of the RgIA4 peptide.

24. The RgIA4 peptide analog of claim 19, wherein the penicillamine-cysteine disulfide bridge reduces one or more of disulfide bridge scrambling, disulfide bridge degradation, or a combination thereof as compared to: an RgIA4 peptide, or an RgIA4 peptide analog without a penicillamine-cysteine disulfide bridge.

25. The RgIA4 peptide analog of claim 19, wherein the penicillamine-cysteine disulfide bridge provides a stability for the RgIA4 peptide analog in human serum that is greater than the stability of an RgIA4 peptide in human serum, wherein the stability in the human serum is measured by the amount of a globular form of the RgIA4 peptide analog remaining after incubation of the RgIA4 peptide analog in 25% human serum AB type and incubated at 37° C. for at least one of 1, 2, 4, 8, 24, 48, or 72 hours.

26. The RgIA4 peptide analog of claim 25, wherein the stability in human serum of the RgIA4 peptide analog is at least one or more of 10%, 20%, 40%, 60%, 80%, 100%, 200%, 300%, 400%, 500%, or 1000% greater than the stability of the RgIA4 peptide in human serum.

27. The RgIA4 peptide analog of claim 19, wherein the RgIA4 peptide analog provides an α9α10 nicotinic acetylcholine receptor selectivity that is substantially equal to the α9α10 nicotinic acetylcholine receptor selectivity of an RgIA4 peptide.

28. The RgIA4 peptide analog of claim 19, wherein the RgIA4 peptide analog provides a α9α10 nicotinic acetylcholine receptor selectivity that is at least one or more of 5×, 10×, 20×, 50×, 100×, or 200×, 500×, or 1,000× more selective for the α9α10 nicotinic acetylcholine receptor compared to a selectivity of a different nicotinic acetylcholine receptor (nAChR) subtype.

29. The RgIA4 peptide analog of claim 28, wherein the different nAChR subtype is selected from the group consisting of: α1β1δϵ, α2β2, α2β4, α3β2, α3β4α4β2, α4β4, α6/α3β2β3, α6/α3β4, α7, or a combination thereof.

30. The RgIA4 peptide analog of claim 19, wherein the RgIA4 peptide analog comprises a C-terminal residue selected from the group consisting of L-citrulline, D-citrulline, L-arginine, D-arginine, L-lysine, D-lysine, L-ornithine, and D-ornithine.

31. The RgIA4 peptide analog of claim 30, wherein the RgIA4 peptide analog provides a solubility that is at least one or more of: 5×, 10×, 20×, 50×, 100×, or 200×, 500×, or 1000× greater than the solubility of the RgIA4 peptide analog without the C-terminal residue.

32. The RgIA4 peptide analog of claim 19, wherein the RgIA4 peptide analog provides a stability in reduced glutathione that is greater than the stability of an RgIA4 peptide in reduced glutathione, wherein the stability in the reduced glutathione is measured by the amount remaining after incubation of 0.1 mg/mL of the RgIA4 peptide analog or the RgIA4 peptide in 10 equivalents of reduced glutathione in phosphate buffered saline (PBS) having a pH of 7.4 and incubated at 37° C. for at least one of 1, 2, 4, 8, 24, 48, or 72 hours.

33. The RgIA4 peptide analog of claim 32, wherein the stability in the reduced glutathione of the @-RgIA4 peptide analog is at least one or more of 10%, 20%, 40%, 60%, 80%, 100%, 200%, 300%, 400%, 500%, or 1000% greater than the stability of the RgIA4 peptide in the reduced glutathione.

34. The RgIA4 peptide analog of claim 19, wherein the RgIA4 peptide analog provides a safety profile that is substantially equal to or greater than the safety profile of an RgIA4 peptide, wherein the safety profile is measured by one or more of:
the analog present in a concentration of 100 μM inhibits less than 25% of the human ether-a-go-go-related gene (hERG) K⁺ channel as measured from an automated-whole cell patch-clamp assay, or
the analog present in a concentration of 10 μM has inhibitory activity of less than 20% as measured in a CYP assay.

35. The RgIA4 peptide analog of claim 19, wherein the RgIA4 peptide analog comprises the amino acid sequence G C (Pen) T D PR C X5 13Y QC B3hY X6 (SEQ ID NO: 10, wherein:
X5 is selected from the group consisting of L-citrulline, D-citrulline, L-arginine or D-arginine, and
X6 is selected from the group consisting of L-arginine or D-arginine.

36. The RgIA4 peptide analog of claim 35, wherein X5 is L-arginine and X6 is L-arginine.

37. The RgIA4 peptide analog of claim 19, wherein the RgIA4 peptide analog comprises the amino acid sequence G C (Pen) T D PR C X5 13Y Q C (3-R-β³hY) X6 (SEQ ID NO: 11), wherein:
X5 is selected from the group consisting of L-citrulline, D-citrulline, L-arginine or D-arginine, and
X6 is selected from the group consisting of L-arginine or D-arginine.

38. The RgIA4 peptide analog of claim 37, wherein X5 is L-arginine and X6 is L-arginine.

39. A composition comprising:
a combination of a therapeutically effective amount of an RgIA4 peptide analog as recited in claim 19 with a pharmaceutically acceptable carrier.

40. (canceled)

41-46. (canceled)

47. A method of maintaining an RgIA4 peptide potency for an α9α10 nicotinic acetylcholine receptor in an RgIA4 peptide analog comprising:
providing an inter-cysteine disulfide bridge between a first cysteine and a second cysteine, and a penicillamine-cysteine disulfide bridge between a first penicillamine and a third cysteine;
providing a β³-homo tyrosine bound to the third cysteine; and
providing a C-terminal residue selected from the group consisting of L-citrulline, D-citrulline, L-arginine, D-arginine, L-lysine, D-lysine, L-ornithine, and D-ornithine.

48-52. (canceled)

53. A method for treating in a subject, a condition that is responsive to α9α10 nicotinic acetylcholine receptor binding, comprising:
administering a therapeutically effective amount of the composition as recited in claim 39 to the subject.

54-73. (canceled)


SEQUENCE LISTING (27 sequences)

SEQ ID NO: 1 - protein, 13 aa, Conus Regius
GCCSDPRCRYRCR

SEQ ID NO: 2 - protein, 13 aa, Artificial Sequence
  Synthetic Construct
  MISC_FEATURE (9)..(9): Citrulline
  MISC_FEATURE (10)..(10): 3-Iodo-tyrosine
GCCTDPRCXXQCY

SEQ ID NO: 3 - protein, 14 aa, Artificial Sequence
  Synthetic Construct
  MISC_FEATURE (3)..(3): L-Pen, D-Pen, L-Cy
  MISC_FEATURE (9)..(9): L-Arg, D-Arg, L-Cit, D-Cit
  MISC_FEATURE (10)..(10): 3-chloro-tyrosine, 3-fluoro-tyrosine, 3-iodo-tyrosine, 3-bromo-tyrosine
  MISC_FEATURE (13)..(13): Any Amino acid other than Cysteine
  MISC_FEATURE (14)..(14): Any Amino acid other than Cysteine
GCXTDPRCXXQCXX

SEQ ID NO: 4 - protein, 14 aa, Artificial Sequence
  Synthetic Construct
  MISC_FEATURE (3)..(3): L-Pen, D-Pen
  MISC_FEATURE (10)..(10): 3-chloro-tyrosine, 3-fluoro-tyrosine, 3-iodo-tyrosine, 3-bromo-tyrosine
  MISC_FEATURE (13)..(13): Any Amino acid other than Cysteine
  MISC_FEATURE (14)..(14): Any Amino acid other than Cysteine
GCXTDPRCRXQCXX

SEQ ID NO: 5 - protein, 14 aa, Artificial Sequence
  Synthetic Construct
  MISC_FEATURE (3)..(3): L-Pen, D-Pen
  MISC_FEATURE (10)..(10): 3-iodo-tyrosine
  MISC_FEATURE (13)..(13): Any Amino acid other than Cysteine
  MISC_FEATURE (14)..(14): Any Amino acid other than Cysteine
GCXTDPRCRXQCXX

SEQ ID NO: 6 - protein, 14 aa, Artificial Sequence
  Synthetic Construct
  MISC_FEATURE (3)..(3): L-Pen, D-Pen
  MISC_FEATURE (10)..(10): 3-iodo-tyrosine
  MISC_FEATURE (13)..(13): beta-3-homo-tyrosine
  MISC_FEATURE (14)..(14): Any Amino acid other than Cysteine
GCXTDPRCRXQCXX

SEQ ID NO: 7 - protein, 14 aa, Artificial Sequence
  Synthetic Construct
  MISC_FEATURE (3)..(3): L-Pen, D-Pen
  MISC_FEATURE (10)..(10): 3-iodo-tyrosine
  MISC_FEATURE (13)..(13): 3-chloro-betahomotyrosine, 3-fluoro-betahomotyrosine, 3-iodo-betahomotyrosine, 3-bromo-betahomotyrosine
  MISC_FEATURE (14)..(14): Any Amino acid other than Cysteine
GCXTDPRCRXQCXX

SEQ ID NO: 8 - protein, 14 aa, Artificial Sequence
  Synthetic Construct
  MISC_FEATURE (3)..(3): L-Pen, D-Pen
  MISC_FEATURE (10)..(10): 3-iodo-tyrosine
  MISC_FEATURE (13)..(13): betahomotyrosine
GCXTDPRCRXQCXR

SEQ ID NO: 9 - protein, 14 aa, Artificial Sequence
  Synthetic Construct
  MISC_FEATURE (3)..(3): L-Pen, D-Pen
  MISC_FEATURE (10)..(10): 3-iodo-tyrosine
  MISC_FEATURE (13)..(13): 3-chloro-betahomotyrosine, 3-fluoro-betahomotyrosine, 3-iodo-betahomotyrosine, 3-bromo-betahomotyrosine
GCXTDPRCRXQCXR

SEQ ID NO: 10 - protein, 14 aa, Artificial Sequence
  Synthetic Construct
  MISC_FEATURE (3)..(3): L-Pen, D-Pen
  MISC_FEATURE (9)..(9): L-Arg, D-Arg, L-Cit, D-Cit
  MISC_FEATURE (10)..(10): 3-iodo-tyrosine
  MISC_FEATURE (13)..(13): beta-3-homo-tyrosine
  MISC_FEATURE (14)..(14): L-Arg, D-Arg
GCXTDPRCXXQCXX

SEQ ID NO: 11 - protein, 14 aa, Artificial Sequence
  Synthetic Construct
  MISC_FEATURE (3)..(3): L-Pen, D-Pen
  MISC_FEATURE (9)..(9): L-Arg, D-Arg, L-Cit, D-Cit
  MISC_FEATURE (10)..(10): 3-iodo-tyrosine
  MISC_FEATURE (13)..(13): 3-chloro-beta-3-homo-tyrosine, 3-fluoro-beta-3-homo-tyrosine, 3-iodo-beta-3-homo-tyrosine, 3-bromo-beta-3-homo-tyrosine
  MISC_FEATURE (14)..(14): L-Arg, D-Arg
GCXTDPRCXXQCXX

SEQ ID NO: 12 - protein, 14 aa, Artificial Sequence
  Synthetic Construct
  MISC_FEATURE (3)..(3): L-Pen, D-Pen
  MISC_FEATURE (10)..(10): 3-iodo-tyrosine
  MISC_FEATURE (13)..(13): beta-3-homo-tyrosine
GCXTDPRCRXQCXR

SEQ ID NO: 13 - protein, 13 aa, Artificial Sequence
  Synthetic Construct
  MISC_FEATURE (2)..(2): L-Pen, D-Pen
  MISC_FEATURE (9)..(9): Citrulline
  MISC_FEATURE (10)..(10): 3-iodo-tyrosine
GXCTDPRCXXQCY

SEQ ID NO: 14 - protein, 13 aa, Artificial Sequence
  Synthetic Construct
  MISC_FEATURE (3)..(3): L-Pen, D-Pen
  MISC_FEATURE (9)..(9): Citrulline
  MISC_FEATURE (10)..(10): 3-iodo-tyrosine
GCXTDPRCXXQCY

SEQ ID NO: 15 - protein, 13 aa, Artificial Sequence
  Synthetic Construct
  MISC_FEATURE (8)..(8): L-Pen, D-Pen
  MISC_FEATURE (9)..(9): Citrulline
  MISC_FEATURE (10)..(10): 3-iodo-tyrosine
GCCTDPRXXXQCY

SEQ ID NO: 16 - protein, 13 aa, Artificial Sequence
  Synthetic Construct
  MISC_FEATURE (9)..(9): Citrulline
  MISC_FEATURE (10)..(10): 3-iodo-tyrosine
  MISC_FEATURE (12)..(12): L-Pen, D-Pen
GCCTDPRCXXQXY

SEQ ID NO: 17 - protein, 13 aa, Artificial Sequence
  Synthetic Construct
  MISC_FEATURE (2)..(2): L-Pen, D-Pen
  MISC_FEATURE (8)..(8): L-Pen, D-Pen
  MISC_FEATURE (9)..(9): Citrulline
  MISC_FEATURE (10)..(10): 3-iodo-tyrosine
GXCTDPRXXXQCY

SEQ ID NO: 18 - protein, 13 aa, Artificial Sequence
  Synthetic Construct
  MISC_FEATURE (3)..(3): L-Pen, D-Pen
  MISC_FEATURE (9)..(9): Citrulline
  MISC_FEATURE (10)..(10): 3-iodo-tyrosine
  MISC_FEATURE (12)..(12): L-Pen, D-Pen
GCXTDPRCXXQXY

SEQ ID NO: 19 - protein, 13 aa, Artificial Sequence
  Synthetic Construct
  MISC_FEATURE (3)..(3): L-Pen, D-Pen
  MISC_FEATURE (10)..(10): 3-iodo-tyrosine
GCXTDPRCRXQCY

SEQ ID NO: 20 - protein, 14 aa, Artificial Sequence
  Synthetic Construct
  MISC_FEATURE (3)..(3): L-Pen, D-Pen
  MISC_FEATURE (10)..(10): 3-iodo-tyrosine
GCXTDPRCRXQCYR

SEQ ID NO: 21 - protein, 14 aa, Artificial Sequence
  Synthetic Construct
  MISC_FEATURE (3)..(3): L-Pen, D-Pen
  MISC_FEATURE (10)..(10): 3-iodo-tyrosine
GCXTDPRCRXQCYR

SEQ ID NO: 22 - protein, 14 aa, Artificial Sequence
  Synthetic Construct
  MISC_FEATURE (3)..(3): L-Pen, D-Pen
  MISC_FEATURE (10)..(10): 3-iodo-tyrosine
  MISC_FEATURE (13)..(13): beta-3-homo-tyrosine
GCXTDPRCRXQCXR

SEQ ID NO: 23 - protein, 14 aa, Artificial Sequence
  Synthetic Construct
  MISC_FEATURE (3)..(3): L-Pen, D-Pen
  MISC_FEATURE (10)..(10): 3-iodo-tyrosine
  MISC_FEATURE (13)..(13): beta-3-homo-tyrosine
GCXTDPRCRXQCXK

SEQ ID NO: 24 - protein, 14 aa, Artificial Sequence
  Synthetic Construct
  MISC_FEATURE (3)..(3): L-Pen, D-Pen
  MISC_FEATURE (10)..(10): 3-iodo-tyrosine
  MISC_FEATURE (13)..(13): beta-3-homo-tyrosine
  MISC_FEATURE (14)..(14): Orn
GCXTDPRCRXQCXX

SEQ ID NO: 25 - protein, 14 aa, Artificial Sequence
  Synthetic Construct
  MISC_FEATURE (3)..(3): L-Pen, D-Pen
  MISC_FEATURE (10)..(10): 3-iodo-tyrosine
  MISC_FEATURE (13)..(13): beta-3-homo-tyrosine
  MISC_FEATURE (14)..(14): Citrulline
GCXTDPRCRXQCXX

SEQ ID NO: 26 - protein, 14 aa, Artificial Sequence
  Synthetic Construct
  MISC_FEATURE (3)..(3): L-Pen, D-Pen
  MISC_FEATURE (10)..(10): 3-iodo-tyrosine
  MISC_FEATURE (13)..(13): beta-3-homo-tyrosine
GCXTDPRCRXQCXE

SEQ ID NO: 27 - protein, 14 aa, Artificial Sequence
  Synthetic Construct
  MISC_FEATURE (10)..(10): 3-iodo-tyrosine
  MISC_FEATURE (13)..(13): beta-3-homo-tyrosine
GCCTDPRCRXQCXR